SEQUENCE LISTING (12 sequences)

SEQ ID NO: 1 - DNA, 27 bp, Artificial Sequence
  Primer useful for BA-05
gaattctttaggattgatagctgtgcc

SEQ ID NO: 2 - DNA, 21 bp, Artificial Sequence
  Primer useful for BA-05
ggtggcgaccatcctccaaaa

SEQ ID NO: 3 - DNA, 888 bp, Artificial Sequence
  BA-14 fusion protein coding sequence
ggatcctctagagtcgacctgcaggcatgcaatgcttattccattaatcaaaaggcttattcaaatacttaccaggagtttactaatattgatcaagcaaaagcttggggtaatgctcagtataaaaagtatggactaagcaaatcagaaaaagaagctatagtatcatatactaaaagcgctagtgaaataaatggaaagctaagacaaaataagggagttatcaatggatttccttcaaatttaataaaacaagttgaacttttagataaatcttttaataaaatgaagacccctgaaaatattatgttatttagaggcgacgaccctgcttatttaggaacagaatttcaaaacactcttcttaattcaaatggtacaattaataaaacggcttttgaaaaggctaaagctaagtttttaaataaagatagacttgaatatggatatattagtacttcattaatgaatgtctctcaatttgcaggaagaccaattattacacaatttaaagtagcaaaaggctcaaaggcaggatatattgaccctattagtgcttttcagggacaacttgaaatgttgcttcctagacatagtacttatcatatagacgatatgagattgtcttctgatggtaaacaaataataattacagcaacaatgatgggcacagctatcaatcctaaagaattcgtgatggaatcccgcaaacgcgcaaggcagacatacacccggtaccagactctagagctagagaaggagtttcacttcaatcgctacttgacccgtcggcgaaggatcgagatcgcccacgccctgtgcctcacggagcgccagataaagatttggttccagaatcggcgcatgaagtggaagaaggagaactga

SEQ ID NO: 4 - protein, 248 aa, Artificial Sequence
  pGEX-4TBA-05 protein sequence for BA-05
GSSRVDLQACNAYSINQKAYSNTYQEFTNIDQAKAWGNAQYKKYGLSKSEKEAIVSYTKSASEINGKLRQNKGVINGFPSNLIKQVELLDKSFNKMKTPENIMLFRGDDPAYLGTEFQNTLLNSNGTINKTAFEKAKAKFLNKDRLEYGYISTSLMNVSQFAGRPIITKFKVAKGSKAGYIDPISAFAGQLEMLLPRHSTYHIDDMRLSSDGKQIIITATMMGTAINPKEFVMNPANAQGRHTPGTRL

SEQ ID NO: 5 - DNA, 37 bp, Artificial Sequence
  Primer
ggatctggttccgcgtcatatgtctagagtcgacctg

SEQ ID NO: 6 - DNA, 32 bp, Artificial Sequence
  Primer
cgcggatccattagttctccttcttccacttc

SEQ ID NO: 7 - skipped

SEQ ID NO: 8 - protein, 247 aa, Artificial Sequence
  pET9a-BA-07 fusion protein
MSRVDLQACNAYSINQKAYSNTYQEFTNIDQAKAWGNAQYKKYGLSKSEKEAIVSYTKSASEINGKLRQNKGVINGFPSNLIKQVELLDKSFNKMKTPENIMLFRGDDPAYLGTEFQNTLLNSNGTINKTAFEKAKAKFLNKDRLEYGYISTSLMNVSQFAGRPIITKFKVAKGSKAGYIDPISAFAGQLEMLLPRHSTYHIDDMRLSSDGKQIIITATMMGTAINPKEFVMNPANAQGRHTPGTRL

SEQ ID NO: 9 - DNA, 783 bp, Artificial Sequence
  BA-05 fusion protein coding sequence
ggatcctctagagtcgacctgcaggcatgcaatgcttattccattaatcaaaaggcttattcaaatacttaccaggagtttactaatattgatcaagcaaaagcttggggtaatgctcagtataaaaagtatggactaagcaaatcagaaaaagaagctatagtatcatatactaaaagcgctagtgaaataaatggaaagctaagacaaaataagggagttatcaatggatttccttcaaatttaataaaacaagttgaacttttagataaatcttttaataaaatgaagacccctgaaaatattatgttatttagaggcgacgaccctgcttatttaggaacagaatttcaaaacactcttcttaattcaaatggtacaattaataaaacggcttttgaaaaggctaaagctaagtttttaaataaagatagacttgaatatggatatattagtacttcattaatgaatgtttctcaatttgcaggaagaccaattattacaaaatttaaagtagcaaaaggctcaaaggcaggatatattgaccctattagtgcttttgcaggacaacttgaaatgttgcttcctagacatagtacttatcatatagacgatatgagattgtcttctgatggtaaacaaataataattacagcaacaatgatgggcacagctatcaatcctaaagaattcgtgatgaatcccgcaaacgcgcaaggcagacatacacccggtaccagactctagagctagagaaggagtttcacttcaatcgctacttga

SEQ ID NO: 10 - protein, 295 aa, Artificial Sequence
  BA-14 fusion protein
GSSRVDLQACNAYSINQKAYSNTYQEFTNIDQAKAWGNAQYKKYGLSKSEKEAIVSYTKSASEINGKLRQNKGVINGFPSNLIKQVELLDKSFNKMKTPENIMLFRGDDPAYLGTEFQNTLLNSNGTINKTAFEKAKAKFLNKDRLEYGYISTSLMNVSQFAGRPIITQFKVAKGSKAGYIDPISAFQGQLEMLLPRHSTYHIDDMRLSSDGKQIIITATMMGTAINPKEFVMESRKRARQTYTRYQTLELEKEFHFNRYLTRRRRIEIAHALCLTERQIKIWFQNRRMKWKKEN

SEQ ID NO: 11 - DNA, 35 bp, Artificial Sequence
  Primer
cctaaagaattcgtgatgaatcccgcaaacgcgca

SEQ ID NO: 12 - DNA, 35 bp, Artificial Sequence
  Primer
tgcgcgtttgcgggattcatcacgaattctttagg

(19) United States
(12) Patent Application Publication
(10) Pub. No.: US 2009/0142325 A1
(43) Pub. Date: Jun. 4, 2009

(54) COMPOSITIONS AND METHODS FOR TREATING TUMOUR SPREADING

(76) Inventors: Dana LASKO, Montreal (CA); Lisa McKerracher, lle des Soeurs Verdun (CA)

Correspondence Address: OGILVY RENAULT LLP, 1981 MCGILL COLLEGE AVENUE, SUITE 1600, MONTREAL, QC, H3A2Y3, CA

(21) Appl. No.: 12/329,918

(22) Filed: Dec. 8, 2008

Related U.S. Application Data
(63) Continuation of application No. 10/902,878, filed on Aug. 2, 2004, now abandoned.
Application No. 10/902,878 is a continuation-in-part of application No. 10/118,079, filed on Apr. 9, 2002, now Pat. No. 6,855,688.
(60) Provisional application No. 60/506,162, filed on Sep. 29, 2003.

(30) Foreign Application Priority Data
Apr. 12, 2001 (CA) 2,342,970
Nov. 13, 2001 (CA) 2,362,004
Jan. 15, 2002 (CA) 2,367,636

(51) Int. Cl. A61K 38/45; A61P 35/00

(52) U.S. Cl. 424/94.5

(57) ABSTRACT

There is provided a method of prevention or inhibition of uncontrolled proliferation of a cancer comprising administration to a mammal of a therapeutically effective amount of a pharmaceutical composition comprising a cell-permeable fusion protein conjugate comprising a polypeptidic cell-membrane transport moiety and a Clostridium botulinum C3 exotransferase unit or a functional analog thereof.

### CROSS-REFERENCE TO RELATED APPLICATIONS

This is a continuation of application Ser. No. 10/902,878 filed on Aug. 2, 2004, which is a continuation in part of application Ser. No. 10/118,079 filed Apr. 9, 2002, which claims priority on Canadian Application 2,342,970; 2,362,004; and 2,367,636 filed Apr. 12, 2001, Nov. 13, 2001 and Jan. 15, 2002, respectively, and further claim priority on U.S. provisional application 60/506,162 filed Sep. 29, 2003. The entire content of these applications is hereby incorporated by reference.

### FIELD OF THE INVENTION

The present invention relates to compositions and methods useful for the treatment of cancer and the prevention of tumor growth related to metastatic cancer. In particular, the present invention relates to compositions comprising a cell-permeable fusion protein conjugate comprising a polypeptidic cell-membrane transport moiety and a Clostridium botulinum C3 exotransferase unit, or a functional analog thereof useful for prevention or inhibition of uncontrolled proliferation and spreading or migration of a metastatic neoplastic cell of a cancer in a mammal.

### BACKGROUND

Cancer in a mammal can be characterized by the uncontrolled division of a population of malignant cells within a tissue in the mammal. If the cell population is localized in a tissue, such uncontrolled division of malignant or cancer cells can lead to the formation of a malignant first tumor in the tissue. If one or more malignant cell or cluster of cells migrates from the site of the localized population to lodge or take root and grow uncontrolled in a second site or in additional tissue sites, which site or sites may be proximal to the first tumor site or which may be remote from the first tumor site, for example in another organ or tissue anatomically distant or distinct from the first tissue, then a second tumor or additional tumors can emerge at the second site or additional sites, respectively, as a result of uncontrolled division of the migrated malignant cell or cells. Migration of one or more malignant cells from the locus of growing cells at the second site to other sites can also occur, and so forth to produce malignant tumors at one or more tissue sites in the mammal. Associated with the growth of such malignant tumors is an often characteristic angiogenesis or process of vascularisation of a tissue proximal to the evolving tumor comprising the development of new capillary blood vessels or in-growth of vasculature and tubular network formation, which new vasculature provides various factors such as nutrients and growth factors that are necessary and permit continued tumor growth.

A tumor is an abnormal mass of tissue that results from excessive cell division that is uncontrolled and progressive, also called a neoplasm. Tumors may be either benign (not cancerous) or malignant.

A variety of methods are presently utilized to treat cancer in a mammal such as man, including for example, surgical procedures in which, for example a tumor and usually some contiguous or proximal non-tumorous tissue is excised from the site of the tumor in a tissue. After removal of the tumor, residual or marginal tissue remains proximal to the site of excision of the tumor in the mammal. If treated with surgery alone however, many patients, particularly those with certain types of cancer, such as cancer selected from the group consisting of breast, brain, colon, skin (melanoma), kidney (renal) and hepatic (liver) cancer will experience recurrence of the cancer in the form of the formation and growth of at least one additional or second tumor, often in the residual margins remaining after excision of the first tumor and sometimes in other tissue or organs and in locations remote or distant from the site of the first tumor. Therefore, in addition to surgery, many cancers are also treated with a combination of therapies, such as those involving administration of cytotoxic chemotherapeutic drugs (e.g., vincristine, vinblastine, cisplatin, methotrexate, 5-FU, etc.) and/or radiation therapy. One difficulty with this approach, however, is that radiotherapeutic and chemotherapeutic agents can be toxic to normal tissues at the dose levels administered, and often create life-threatening side effects in the patient. These cancer therapies can often have high failure/remission rates which can result in death of the patient. Some more recent therapeutic treatments take advantage of dysregulation of cellular signaling by altered or upregulated gene products in cancer cells, such a the use of tamoxifen for breast cancer and Gleevec® (imatinib mesylate from Novaitis) for chronic myeloid leukemia (also referred to as CML).

An additional difficulty of present methods is that local recurrence and local disease control remains a major challenge in the treatment of malignancy. Over 600,000 patients annually (in the U.S.) have localized malignant disease (with no evidence of distant metastatic spread) at the time of presentation, representing about 64% of all patients diagnosed with malignancy but not including nonmelanoma skin cancer or carcinoma in situ. For a majority of these patients, surgical resection of the disease represents the greatest chance for a cure, and over 400,000 patients will be cured after the initial treatment. Unfortunately, about 200,000 (or about one third of all patients with localized disease) will relapse after the initial treatment. Of those who relapse, the number who will relapse due to local recurrence of the disease can amount to about 133,000 patients annually (or about 21% of all those with localized disease). The number who will relapse due to distant metastases of the disease is about 68,000 patients annually (or about 11% of all those with localized disease). About another 100,000 patients annually will die as a direct result of an inability to control the local growth of the disease.

Brain tumors are an especially deadly form of cancer. About one third of all primary gliomas (gliomas represent about ⅓ of all brain tumors) are fatal, and the mean survival for glioma patients is about 10 to about 12 months. The five year survival rate is about 9%. Gliomas are neuroectodermal tumors of neuroglial origin, and include astrocytoma derived from astrocytes, oligodendroglioma derived from oligodendrocytes, and ependymoma derived from ependymal cells. A number of studies suggest that combination therapies will needed to treat these aggressive tumors. The most common type of brain tumor arises by metastasis, and there are about 100,000 to about 170,000 brain tumors diagnosed per year in the USA. The mean survival time ranges from about 2.9 months to about 3.4 months. Metastatic brain tumors are mainly treated with radiosurgery or tumor resection. Better outcomes have been reported when surgery is combined with radiation than with radiation alone. The most common origins for metastatic tumors to the brain comprise mammary cancers, bronchial cancers, gastrointestinal carcinoma, renal carcinoma, and malignant melanoma. Metastatic brain tumors may be may be clinically explosive, especially after removal of a primary tumor. Individuals suspected of having CNS cancer (which includes brain tumors and brain cancer as used herein) may be identified by detecting clinical symptoms such as headache, nausea or vomiting, seizures, altered mental status, altered speech, visual abnormalities, and/or paralysis. A method of inhibiting metastases of a primary CNS cancer in a mammal is also within the scope of the present invention.

### Angiogensis

Many of the mechanisms which control angiogenesis in normal tissues are altered in the presence of a malignant tumors during tumor growth. The formation and metastasis of a tumor involved pathological angiogenesis. Like healthy tissues, a tumor requires connection to blood vessels in order to receive nutrients and oxygen and to eliminate cellular wastes. Thus, pathological angiogenesis is critical to the growth and expansion of tumors. Tumors in which angiogenesis is important include solid malignant tumors as well as benign tumors, for example such as acoustic neuroma, neurofibroma, trachoma and pyogenic granulomas. In metastasis, pathological angiogenesis is important in at least two aspects. The formation of blood vessels in tumors allows tumor cells to enter the blood stream and to circulate throughout the body. Angiogenesis supports the formation and growth of new tumors seeded by tumor cells that have left the primary site or first tumor as used herein.

Angiogenesis is the complex process of blood vessel formation. The process involves both biochemical and cellular events, including (1) activation of endothelial cells (ECs) by an angiogenic stimulus; (2) degradation of the extracellular matrix, invasion of the activated ECs into the surrounding tissues, and migration toward the source of the angiogenic stimulus; and (3) proliferation and differentiation of ECs to form new blood vessels (Folkman et al., 1991, J. Biol. Chem. 267:10931-10934).

The control of angiogenesis is a highly regulated process involving angiogenic stimulators and inhibitors. In healthy humans and animals, angiogenesis occurs under specific, restricted situations. For example, angiogenesis is normally observed in fetal and embryonal development, development and growth of normal tissues and organs, wound healing, and the formation of the corpus luteum, endometrium and placenta.

Another embodiment of the present invention comprises the inhibition of angiogenesis by a cell-permeable fusion protein conjugate comprising a polypeptidic cell-membrane transport moiety and a Clostridium botulinum C3 exotransferase unit, or a functional analog thereof, for example a fusion protein such as BA-05.

Another embodiment of the present invention comprises the inhibition of angiogenesis by an effective amount of a pharmaceutical composition comprising a cell-permeable fusion protein conjugate comprising a polypeptidic cell-membrane transport moiety and a Clostridium botulinum C3 exotransferase unit, or a functional analog thereof, for example a fusion protein such as BA-05.

### Rho Signaling and Cancer

Rho (also known as Ras homology) family proteins have been investigated in relation to cancer. Ras (and RhoB as a secondary target) are targets for metastasis by molecules that inhibit posttranslational modification. However, these therapeutics investigations focus on Ras and are limited to RhoB among Rho family members, whereas the current invention has the potential to affect signaling of RhoA, RhoB, and RhoC. RhoA, RhoB and Rho C are Rho family members specifically inhibited by the fusion protein BA-05. In some studies, C3 exoenzyme has been used as a molecular probe for Rho involvement and significant changes have been found in parameters of in vitro models considered important in cancer such as cell transformation. In such studies C3 was applied by methods ranging from prolonged incubation in the tissue culture medium to heterologous gene expression. It is an advantage of the current invention that compositions and methods of the current invention such as BA-05 and administration of BA-05 offer a significant advantage compared to C3 because of the ability of compositions of the current invention to penetrate inside tumor cells to inactivate rapidly Rho at lower doses. In another advantage, the current invention provides compositions comprising a fusion protein of this invention such as BA-07, which fusion protein has the ability to penetrate both tumour cells and endothelial cells that in the absence of the fusion protein can form new blood vessels that supply tumor growth.

Mutations in Rho family regulatory proteins have been found in clinical samples of malignancies. Examples include the DLC1 gene in hepatocellular carcinoma; p-190-A, in a genomic region that is altered in gliomas and astrocytomas; GRAF, which has loss of function mutations in leukemia; and LARG, found in some a gene fusions found in acute myeloid leukemia. Genetically engineered point mutations activate RhoA and induce cellular transformation in vitro.

### Rho Family Gene Expression in Human Malignancy

The small GTPase Rho is a cellular target of BA-05, and is up-regulated in certain cancers, such as malignant melanoma and breast cancer.

In contrast to the small GTPase Ras, Rho GTPases have not been identified as oncogenes by traditional approaches, although evidence has accumulated for dysregulation of Rho gene expression in cancer. For instance, increased levels of RhoA mRNA have been observed in testicular germ cell tumor, and increased RhoC mRNA in inflammatory breast cancer and pancreatic adenocarcinoma.

The Cancer Genome Anatomy Project (CGAP) correlates gene expression with site of malignancy. Data is available on transcription levels in libraries made from malignant and normal cells (NCBI, 2002). Transcription levels are measured using “tags”, i.e., 10 base oligonucleotides that uniquely define a gene. Available data on RhoA, RhoB and RhoC I shows upregulation of RhoA and to a lesser extent in these measurements, RhoC in malignancies of the brain and in the breast. Rho A sequence tags are found more often in libraries made from malignancies of the cerebellum and breast than from the corresponding normal tissue. Expression levels were elevated in glioblastoma but not in astrocytoma. The result for astrocytoma corresponds with reduction in RhoA protein levels in astrocytic tumor samples. Rho C mRNA is overexpressed in breast malignancies and to a slight extent in some brain malignancies, and may be downregulated in colon adenocarcinoma.

However, relative levels of Rho cDNA in such libraries may not directly relate to Rho action in the cell, which undergoes complex regulation involving numerous other gene products.

### Rho Proteins in Tumors and Tumor Cell Lines

Rho protein expression has been investigated at several tumor sites in humans. Increased protein levels are found in colon, breast and lung tumors. RhoA and RhoB levels have been found in 5 μm sections from head and neck squamous cell carcinomas using polyclonal antibodies directed against these proteins, followed by visualization using a VectaStain kit (Vector Labs) and image analysis. Nearby “nonneoplastic” areas were used as controls. Although Rho A protein levels increased with tumor progression, RhoB levels decreased in invasive tumors compared to carcinomas in situ and well-differentiated tumors. Activation states were not studied.

Overexpression of RhoA and RhoB may occur in breast and lung adenocarcinomas compared to normal tissue, whereas expression of Rho proteins is decreased in astrocytic tumors and inversely related to grade II to IV malignancy.

### Rho and Metastasis

Rho is involved in regulation of cell migration and motility. MM1 rat hepatoma cells transfected with Rho A mutant constructs (Val¹⁴ or Val¹⁴Ile⁴¹) result in constitutively activated Rho. In an in vitro invasion assay, the percent of seeded cells capable of infiltration into a mesothelial cell layer was correlated with the level of expression of transfected RhoA Val¹⁴. When these activated RhoA-transfected cells were used in an in vivo assay in the peritoneal cavity, 6 of 10 implants resulted in tumor nodules compared with 2 of 8 for mock transfectants. These results indicate that active Rho is correlated with tumorigenicity.

A comprehensive study of gene expression compared two metastatic melanoma model systems, one human and one mouse, and looked at the shared similarities in gene expression by microarray concluded that RhoC expression was altered in increasing levels of metastasis (Clark et al., 2000). Furthermore, when gene expression was manipulated experimentally, RhoC overexpression induced a human melanoma cell line to switch from low metastatic potential to high metastatic potential. Although RhoA was not observed to be overexpressed, a dominant negative mutation (N19RhoA) diminished metastatic potential.

A set of 70 genes whose expression correlated with propensity for metastasis in human breast cancer was identified (van't Veer et al., 2002). Although Rho genes were not found, the value of a disease marker as a prognostic indicator is not necessarily related to its value as a target for therapy. In the case of Rho family signaling, there is complex regulation of enzymatic activity and protein-protein interactions which is not apparent from measurements of transcription levels alone.

### SUMMARY OF THE INVENTION

Individual fusion proteins of this invention are sometimes referred to by designations such as BA-05, BA-07, and the like.

This invention discloses a method of prevention or inhibition of uncontrolled proliferation and spreading or migration of a metastatic neoplastic cell of a cancer in a mammal, comprising administration to the mammal of a therapeutically effective amount of a pharmaceutical composition comprising a cell-permeable fusion protein conjugate comprising a polypeptidic cell-membrane transport moiety and a Clostridium botulinum C3 exotransferase unit, or a functional analog thereof.

This invention discloses a method of prevention or inhibition of uncontrolled proliferation and spreading or migration, within a resection margin of a host tissue proximal to the site of excision of a tumor of a cancer in a mammal, of a metastatic neoplastic cell residing in the resection margin, comprising administration of a therapeutically effective amount of a pharmaceutical composition comprising a cell-permeable fusion protein conjugate comprising a polypeptidic cell-membrane transport moiety and a Clostridium botulinum C3 exotransferase unit, or a functional analog thereof, said administration being directly on to the surface of the resection margin or below the surface of the resection margin or into the tissue proximal to the resection margin which remains in the mammal, said administration in a time interval prior to or subsequent to or prior to and subsequent to excision or removal of the tumor.

This invention discloses a method of prevention of growth of a tumor from a malignant cell in a host tissue in a mammal comprising administration to the mammal of a therapeutically effect amount of a pharmaceutical composition comprising a cell-permeable fusion protein conjugate comprising a polypeptidic cell-membrane transport moiety and a Clostridium botulinum C3 exotransferase unit, or a functional analog thereof, wherein the fusion protein simultaneously prevents or inhibits at least two of malignant cell migration, malignant cell proliferation, angiogenesis or tubular structure formation or capillary network growth proximal to the malignant cell, and secretion of an active metalloproteinase from the malignant cell.

This invention discloses a method of prevention of growth within a resection margin of a host tissue proximal to a site of excision or removal of a first tumor of a cancer in a mammal, of a second tumor comprising a residual tumor cell of the cancer, the method comprising administration of a therapeutically effective amount of a pharmaceutical composition comprising a cell-permeable fusion protein conjugate comprising a polypeptidic cell-membrane transport moiety and a Clostridium botulinum C3 exotransferase unit, or a functional analog thereof, said administration being directly on to the surface of the resection margin or below the surface of the resection margin or into the tissue proximal to the resection margin which remains in the mammal, and said administration being in a time interval prior to, or subsequent to, or both prior to and subsequent to excision or removal of the first tumor, wherein the fusion protein simultaneously prevents or inhibits at least two of residual tumor cell migration, residual tumor cell proliferation, angiogenesis or tubular structure formation or capillary network growth proximal to the residual tumor cell, and secretion of an active metalloproteinase from the residual tumor cell.

In one aspect, the present invention comprises a method of inhibiting metastases of a systemic cancer into the CNS (central nervous system) of a mammal comprising administration to the mammal of a therapeutically effective amount of a pharmaceutical composition comprising a cell-permeable fusion protein conjugate comprising a polypeptidic cell-membrane transport moiety and a Clostridium botulinum C3 exotransferase unit, or a functional analog thereof, for example a fusion protein such as BA-05.

In one aspect, a therapeutically effective amount of a pharmaceutical composition comprising a cell-permeable fusion protein conjugate comprising a polypeptidic cell-membrane transport moiety and a Clostridium botulinum C3 exotransferase unit, or a functional analog thereof, for example a fusion protein such as BA-05, can exhibit anti-angiogenic activity and is useful in the treatment of cancer.

In one aspect, this invention discloses a method of prevention or inhibition of uncontrolled proliferation and spreading or migration of a metastatic neoplastic cell of a cancer in a mammal, comprising administration to the mammal of a therapeutically effective amount of a pharmaceutical composition comprising a cell-permeable fusion protein conjugate comprising a polypeptidic cell-membrane transport moiety and a Clostridium botulinum C3 exotransferase unit, or a functional analog thereof.

In a second aspect, this invention discloses a method of prevention or inhibition of uncontrolled proliferation and spreading or migration, within a resection margin of a host tissue proximal to the site of excision of a tumor of a cancer in a mammal, of a metastatic neoplastic cell residing in the resection margin, comprising administration of a therapeutically effective amount of a pharmaceutical composition comprising a cell-permeable fusion protein conjugate comprising a polypeptidic cell-membrane transport moiety and a Clostridium botulinum C3 exotransferase unit, or a functional analog thereof, said administration being directly on to the surface of the resection margin or below the surface of the resection margin or into the tissue proximal to the resection margin which remains in the mammal, said administration in a time interval prior to or subsequent to or prior to and subsequent to excision or removal of the tumor.

In a third aspect, this invention discloses a method of prevention of growth of a tumor from a malignant cell in a host tissue in a mammal comprising administration to the mammal of a therapeutically effect amount of a pharmaceutical composition comprising a cell-permeable fusion protein conjugate comprising a polypeptidic cell-membrane transport moiety and a Clostridium botulinum C3 exotransferase unit, or a functional analog thereof, wherein the fusion protein simultaneously prevents or inhibits at least two of malignant cell migration, malignant cell proliferation, angiogenesis or tubular structure formation or capillary network growth proximal to the malignant cell, and secretion of an active metalloproteinase from the malignant cell.

In a fourth aspect, this invention discloses a method of prevention of growth within a resection margin of a host tissue proximal to a site of excision or removal of a first tumor of a cancer in a mammal, of a second tumor comprising a residual tumor cell of the cancer, the method comprising administration of a therapeutically effective amount of a pharmaceutical composition comprising a cell-permeable fusion protein conjugate comprising a polypeptidic cell-membrane transport moiety and a Clostridium botulinum C3 exotransferase unit, or a functional analog thereof, said administration being directly on to the surface of the resection margin or below the surface of the resection margin or into the tissue proximal to the resection margin which remains in the mammal, and said administration being in a time interval prior to, or subsequent to, or both prior to and subsequent to excision or removal of the first tumor, wherein the fusion protein simultaneously prevents or inhibits at least two of residual tumor cell migration, residual tumor cell proliferation, angiogenesis or tubular structure formation or capillary network growth proximal to the residual tumor cell, and secretion of an active metalloproteinase from the residual tumor cell.

In a fifth aspect, this invention discloses a use of a pharmaceutical composition comprising a cell-permeable fusion protein conjugate comprising a polypeptidic cell-membrane transport moiety and a Clostridium botulinum C3 exotransferase unit, or a functional analog thereof, in the manufacture of a medicine for the prevention or inhibition of uncontrolled proliferation and spreading or migration of a metastatic neoplastic cell of a cancer in a mammal.

In a sixth aspect, this invention discloses a use of a pharmaceutical composition comprising a cell-permeable fusion protein conjugate comprising a polypeptidic cell-membrane transport moiety and a Clostridium botulinum C3 exotransferase unit, or a functional analog thereof, in the manufacture of a medicine for the prevention or inhibition of uncontrolled proliferation and spreading or migration, within a resection margin of a host tissue proximal to the site of excision of a tumor of a cancer in a mammal, of a metastatic neoplastic cell residing in the resection margin, suitable for administration directly on to the surface of the resection margin or below the surface of the resection margin or into the tissue proximal to the resection margin which remains in the mammal, in a time interval prior to or subsequent to or prior to and subsequent to excision or removal of the tumor.

In a seventh aspect, this invention discloses a use of a pharmaceutical composition comprising a cell-permeable fusion protein conjugate comprising a polypeptidic cell-membrane transport moiety and a Clostridium botulinum C3 exotransferase unit, or a functional analog thereof, in the manufacture of a medicine for prevention of growth of a tumor from a malignant cell in a host tissue in a mammal, wherein the fusion protein simultaneously prevents or inhibits at least two of malignant cell migration, malignant cell proliferation, angiogenesis or tubular structure formation or capillary network growth proximal to the malignant cell, and secretion of an active metalloproteinase from the malignant cell.

In an eighth aspect, this invention discloses a use of a pharmaceutical composition comprising a cell-permeable fusion protein conjugate comprising a polypeptidic cell-membrane transport moiety and a Clostridium botulinum C3 exotransferase unit, or a functional analog thereof, in the manufacture of a medicine for prevention of growth within a resection margin of a host tissue proximal to a site of excision or removal of a first tumor of a cancer in a mammal, of a second tumor comprising a residual tumor cell of the cancer, by administration directly on to the surface of the resection margin or below the surface of the resection margin or into the tissue proximal to the resection margin which remains in the mammal in a time interval prior to, or subsequent to, or both prior to and subsequent to excision or removal of the first tumor, wherein the fusion protein simultaneously prevents or inhibits at least two of residual tumor cell migration, residual tumor cell proliferation, angiogenesis or tubular structure formation or capillary network growth proximal to the residual tumor cell, and secretion of an active metalloproteinase from the residual tumor cell.

In a ninth aspect, this invention discloses another, aspect of the previous aspects, wherein the fusion protein conjugate is BA-05.

In a tenth aspect, this invention discloses another aspect of the previous aspects, wherein the cancer is selected from the group consisting of breast, brain, colon, skin, kidney, and hepatic cancer.

In an eleventh aspect, this invention discloses another aspect of the previous aspects, wherein the cancer is a brain tumor selected from the group consisting of glial tumors, neuron tumors, pineal gland tumors, menigeal tumors, tumors of nerve sheath, lymphomas, malformative tumors, and metastatic tumors located in the brain derived from tumors of the lung, breast, melanoma, kidney, and gastrointestinal tract.

In a twelfth aspect, this invention discloses another aspect of the previous aspects, wherein the cancer is a brain tumor selected from the group consisting of anaplastic astrocytoma, glioblastoma multiform, pilocytic astrocytoma, oligodendroglioma, ependymoma, myxopapillary ependymoma, subependymoma, choroid plexus papilloma, neuroblastoma, ganglioneuroblastoma, ganglioneuroma, and medulloblastoma, pineoblastoma and pineocytoma, meningioma, meningeal hemangiopericytoma, meningeal sarcoma, Schwannoma (neurolemmoma) and neurofibroma, Hodgkin's lymphoma, non-Hodgkin's lymphoma, primary and secondary subtypes of Hodgkin's lymphoma, primary and secondary subtypes of non-Hodgkin's lymphoma, craniopharyngioma, epidermoid cysts, dermoid cysts and colloid cysts.

In a thirteenth aspect, this invention discloses another aspect of the previous aspects, wherein the therapeutically effective amount is about 0.001 micrograms per cc to about 50 micrograms per cc of tissue.

In a fourteenth aspect, this invention discloses another aspect of the previous aspects, wherein the therapeutically effective amount is about 0.0001 micrograms of fusion protein per cubic centimeter (cc) of tissue to about 100 micrograms per cubic centimeter of tissue.

In a fifteenth aspect, this invention discloses another aspect of the previous aspects, wherein the therapeutically effective amount is about 1 micrograms per milliliter to about 10 micrograms per milliliter to about 50 micrograms per milliliter.

In a sixteenth aspect, this invention discloses another aspect of the previous aspects, wherein the administration is by injection, by topical application, or by implantation.

In a seventeenth aspect, this invention discloses another aspect of the previous aspects, wherein the administration is selected from the group consisting of intrarticular, intraocular, intranasal, intraneural, intradermal, intraosteal, sublingual, oral, topical, intravesical, intrathecal, intravenous, intraperitoneal, intracranial, intramuscular, subcutaneous, inhalation, atomization and inhalation, application directly into a tumor, application directly into a disease site, application directly on or into the margins remaining after resection of a tumor, enteral, enteral together with a gastroscopic procedure, and ECRP.

In an eighteenth aspect, this invention discloses another aspect of the previous aspects, wherein the polypeptidic cell-membrane transport moiety comprises a peptide containing from about 5 to about 50 amino acids.

In a nineteenth aspect, this invention discloses another aspect of the previous aspects, wherein the Clostridium botulinum Ce exotransferase unit comprises the amino acid sequence designated by the sequence of fusion protein BA-05.

In a twentieth aspect, this invention discloses another aspect of the previous aspects, wherein the functional analog comprises a protein exhibiting activity in the range of 50% to 500% of that of wild type Clostridium botulinum Ce exotransferase.

In a twenty-first aspect, this invention discloses another aspect of the previous aspects, wherein the pharmaceutical composition comprises a pharmaceutically acceptable carrier.

In a twenty-second aspect, this invention discloses another aspect of the previous aspects, wherein the pharmaceutical composition comprises a pharmaceutically acceptable carrier selected from the group consisting of poly(ethylene-co-vinyl acetate), PVA, partially hydrolyzed poly(ethylene-co-vinyl acetate), poly(ethylene-co-vinyl acetate-co-vinyl alcohol), a cross-linked poly(ethylene-co-vinyl acetate), a cross-linked partially hydrolyzed poly(ethylene-co-vinyl acetate), a cross-linked poly(ethylene-co-vinyl acetate-co-vinyl alcohol), poly-D,L-lactic acid, poly-L-lactic acid, polyglycolic acid, PGA, copolymers of lactic acid and glycolic acid, polycaprolactone, polyvalerolactone, poly (anhydrides), copolymers of polycaprolactone with polyethylene glycol, copolymers of polylactic acid with polyethylene glycol, polyethylene glycol; and combinations and blends thereof.

In a twenty-third aspect, this invention discloses another aspect of the previous aspects, wherein the pharmaceutical composition comprises a pharmaceutically acceptable carrier comprising an aqueous gelatin, an aqueous protein, a polymeric carrier, a cross-linking agent, and a combination thereof.

In a twenty-fourth aspect, this invention discloses another aspect of the previous aspects, wherein the pharmaceutical composition comprises a pharmaceutically acceptable carrier comprising a matrix.

In a twenty-fifth aspect, this invention discloses another aspect of the previous aspects, wherein the pharmaceutical composition comprises a pharmaceutically acceptable carrier comprising water, a pharmaceutically acceptable buffer salt, a pharmaceutically acceptable buffer solution a pharmaceutically acceptable antioxidant, ascorbic acid, one or more low molecular weight pharmaceutically acceptable polypeptide, a peptide comprising about 2 to about 10 amino acid residues, one or more pharmaceutically acceptable protein, one or more pharmaceutically acceptable amino acid, an essential-to-human amino acid, one or more pharmaceutically acceptable carbohydrate, one or more pharmaceutically acceptable carbohydrate-derived material, a non-reducing sugar, glucose, sucrose, sorbitol, trehalose, mannitol, maltodextrin, dextrins, cyclodextrin, a pharmaceutically acceptable chelating agent, EDTA, DTPA, a chelating agent for a divalent metal ion, a chelating agent for a trivalent metal ion, glutathione, pharmaceutically acceptable nonspecific serum albumin, and combinations thereof.

In a twenty-sixth aspect, this invention discloses another aspect of the previous aspects, wherein the pharmaceutical composition is sterile.

In a twenty-seventh aspect, this invention discloses another aspect of the previous aspects, wherein the pharmaceutical composition is sterilizable.

In a twenty-eighth aspect, this invention discloses another aspect of the previous aspects, wherein the pharmaceutical composition is sterilized.

In a twenty-ninth aspect, this invention discloses another aspect of the previous aspects, wherein the pharmaceutical composition is in a vial in a unit dosage amount or in an integral multiple of a unit dosage amount.

In a thirtieth aspect, this invention discloses another aspect of the previous aspects, wherein the pharmaceutical composition is dried.

In a thirty-first aspect, this invention discloses another aspect of the previous aspects, wherein the pharmaceutical composition comprises a dehydrated matrix.

In a thirty-second aspect, this invention discloses another aspect of the previous aspects, wherein the pharmaceutical composition comprises a pharmaceutically acceptable carrier.

In a thirty-third aspect, this invention discloses another aspect of the previous aspects, wherein the pharmaceutical composition comprises a fusion protein in a lyophilized matrix.

### Antagonism of Rho and Apoptosis

Mechanisms to control cell proliferation are dysregulated in cancer. An increased apoptosis in EL4 Murine T lymphoma cells occurs after Rho inactivation by recombinant C3 exoenzyme. In NIH3t3 cells, treatment with the Rho kinase inhibitor Y-27632 significantly inhibited anchorage-independent growth. In one embodiment, inactivation of Rho can prevent tumour cell proliferation, and the present invention comprises the reduction or arrest of cell proliferation, or induction of apoptosis by a cell-permeable fusion protein conjugate comprising a polypeptidic cell-membrane transport moiety and a Clostridium botulinum C3 exotransferase unit, or a functional analog thereof, for example a fusion protein such as BA-07. In another embodiment, the present invention comprises the reduction or arrest of cell proliferation, or induction of apoptosis by an effective amount of a pharmaceutical composition comprising a cell-permeable fusion protein conjugate comprising a polypeptidic cell-membrane transport moiety and a Clostridium botulinum C3 exotransferase unit, or a functional analog thereof, for example a fusion protein such as BA-07.

### Antagonism of Rho and Cell Migration.

Metastatic cancer cells are highly migratory. Inactivation of Rho can prevent cell migration in certain cell types. C3 transferase and the Rho kinase inhibitor Y-27632 block cellular invasion by HT29 human colon cancer cells. In a v-Crk-inducible rat fibroblast 3Y1 cell line, C3 and Y-27632 inhibited v-Crk, resulting in decreased cell motility. Decreased apoptosis in RhoB−/− cells in Rho B+/− or RhoB−/− MEF cells treated with doxorubicin, radiation or Taxol results from the lack of RhoB protein. In another embodiment, antagonism of Rho can reduce cell migration and metastasis, and the present invention comprises the inhibition of cell migration by a cell-permeable fusion protein conjugate comprising a polypeptidic cell-membrane transport moiety and a Clostridium botulinum C3 exotransferase unit, or a functional analog thereof, for example a fusion protein such as BA-07.

Another embodiment of the present invention comprises the inhibition of cell migration by an effective amount of a pharmaceutical composition comprising a cell-permeable fusion protein conjugate comprising a polypeptidic cell-membrane transport moiety and a Clostridium botulinum C3 exotransferase unit, or a functional analog thereof, for example a fusion protein such as BA-05.

### Antagonism of Rho and Matrix Metalloproteinases (MMPs)

Invasive tumour cells have the property of being able to degrade the extracellular matrix that surround them by secreting proteases that degrade the extracellular matrix. One important class of proteases that are secreted by tumour cells is the matrix metalloproteinases (MMPs). These enzymes open up paths in the matrix through which the cancer cells can invade and spread. Tumour cells can produce different types of MMPs, and MMP are often made as pro-enzymes that are cleaved and released upon activation. MMP1 cleaves collagen matrix. MMP-2 may play an important role invasion of lung cancer cells. MMP-9 has also been implicated in tumour cell invasion. In another embodiment, the present invention comprises the inhibition of MMP expression, MMP processing or MMP secretion from a tumor cell, the inhibition by a cell-permeable fusion protein conjugate comprising a polypeptidic cell-membrane transport moiety and a Clostridium botulinum C3 exotransferase unit, or a functional analog thereof, for example a fusion protein such as BA-07.

In another embodiment, the present invention comprises the inhibition of MMP expression, MMP processing or MMP secretion by an effective amount of a pharmaceutical composition comprising a cell-permeable fusion protein conjugate comprising a polypeptidic cell-membrane transport moiety and a Clostridium botulinum C3 exotransferase unit, or a functional analog thereof, for example a fusion protein such as BA-05.

### BA-05 and BA-07 as Rho Antagonists

BA-05 and BA-07 are genetically engineered forms of C3 exoenzyme. C3 exoenzyme is a bacteriophage-derived secreted protein discovered in some strains of Clostridium botulinum that transfers an ADP-ribose group to an asparagine residue of the small regulatory GTPases, RhoA, RhoB and RhoC. C3 inactivates Rho because ADP-ribosylation prevents activation of Rho. Novel modifications that distinguish BA-05 and BA-07 include a C-terminal transport peptide that allows efficient entry into the cytoplasm, resulting in a more potent Rho antagonist. BA-05 and BA-07 differ in silent mutations in the non-enzymatic region. BA-07 allows expression in a commercial-scale vector for purification of the protein useful as a therapeutic drug. In one aspect of this invention, a fusion protein such as BA-07 can be considered to be a cell permeable disrupter of protein-protein interactions important in signal transduction.

The present invention provides BA-05 and BA-07 variants such as a cell-permeable fusion protein conjugate comprising a polypeptidic cell-membrane transport moiety whose amino acid sequence can be varied or shorted or elongated or truncated to comprise a variant of BA-05 and a Clostridium botulinum C3 exotransferase unit whose amino acid sequence can be varied, elongated, shorted, or truncated in a variant, or a functional analog thereof, as anti-neoplastic and anti-metastatic compositions, as well as methods and devices which utilize such compositions for the treatment of cancer and other malignant diseases.

Within one aspect of the present invention, compositions and methods are provided to alter BA-07 DNA sequence expressed in a plasmid to enhance the ability to purify large amounts of BA-07 for formulation in a pharmaceutically acceptable carrier safe for therapeutic use.

BA-05 and BA-07 are Fusion Proteins According to this Invention.

Included in this invention are variants of BA-05 that retain a proline-rich transport sequence and enough of the C3 transferase unit to retain enzymatic activity to ADP ribosylsate Rho.

In accordance with the present invention a conjugate or fusion protein comprising a therapeutically active agent is provided whereby the active agent may be delivered across a cell wall membrane, the conjugate or fusion protein comprising a transport subdomain(s) or moiety(ies) in addition to an active agent moiety(ies). More particularly, in accordance with the present invention a therapeutically active agent as conjugate or fusion protein is provided comprising a polypeptidic cell-membrane transport moiety and a Clostridium botulinum C3 exotransferase unit as a therapeutically active unit, or a functional analog thereof, wherein the therapeutically active agent can inhibit tumor cell migration, promote apoptosis of tumor cells, inhibit angiogenesis, and inhibit production of metalloproteinases associated with tumor growth.

It is an advantage that the compositions and methods of the present invention provide a significant improvement over previous drugs designed to arrest tumor spread or metastasis because a single compound of the invention can act as a combination therapy to arrest several very different aspects of tumor growth and spread. It is an advantage of that a composition of the present invention, such as a composition comprising BA-07, can prevent or retard or inhibit: tumor cell migration, tumor cell proliferation, angiogenesis at a tumor site, and the secretion of active metalloproteinases. It is an advantage of the present invention that pharmaceutically active compounds can penetrate a cancer cell without reliance on a receptor-based membrane transport mechanism. It is an advantage of the present invention that pharmaceutically active compounds can inactivate members of the Rho family GTPases. It is an advantage of the present invention that pharmaceutically active compounds are Rho antagonists.

This invention discloses a method of prevention or inhibition of uncontrolled proliferation and spreading or migration of a metastatic neoplastic cell of a cancer in a mammal, comprising administration to the mammal of a therapeutically effective amount of a pharmaceutical composition comprising a cell-permeable fusion protein conjugate comprising a polypeptidic cell-membrane transport moiety and a Clostridium botulinum C3 exotransferase unit, or a functional analog thereof.

This invention discloses a method of prevention or inhibition of uncontrolled proliferation and spreading or migration, within a resection margin of a host tissue proximal to the site of excision of a tumor of a cancer in a mammal, of a metastatic neoplastic cell residing in the resection margin, comprising administration of a therapeutically effective amount of a pharmaceutical composition comprising a cell-permeable fusion protein conjugate comprising a polypeptidic cell-membrane transport moiety and a Clostridium botulinum C3 exotransferase unit, or a functional analog thereof, said administration being directly on to the surface of the resection margin or below the surface of the resection margin or into the tissue proximal to the resection margin which remains in the mammal, said administration in a time interval prior to or subsequent to or prior to and subsequent to excision or removal of the tumor.

This invention discloses a method of prevention of growth of a tumor from a malignant cell in a host tissue in a mammal comprising administration to the mammal of a therapeutically effect amount of a pharmaceutical composition comprising a cell-permeable fusion protein conjugate comprising a polypeptidic cell-membrane transport moiety and a Clostridium botulinum C3 exotransferase unit, or a functional analog thereof, wherein the fusion protein simultaneously prevents or inhibits at least two of malignant cell migration, malignant cell proliferation, angiogenesis or tubular structure formation or capillary network growth proximal to the malignant cell, and secretion of an active metalloproteinase from the malignant cell.

This invention discloses a method of prevention of growth within a resection margin of a host tissue proximal to a site of excision or removal of a first tumor of a cancer in a mammal, of a second tumor comprising a residual tumor cell of the cancer, the method comprising administration of a therapeutically effective amount of a pharmaceutical composition comprising a cell-permeable fusion protein conjugate comprising a polypeptidic cell-membrane transport moiety and a Clostridium botulinum C3 exotransferase unit, or a functional analog thereof, said administration being directly on to the surface of the resection margin or below the surface of the resection margin or into the tissue proximal to the resection margin which remains in the mammal, and said administration being in a time interval prior to, or subsequent to, or both prior to and subsequent to excision or removal of the first tumor, wherein the fusion protein simultaneously prevents or inhibits at least two of residual tumor cell migration, residual tumor cell proliferation, angiogenesis or tubular structure formation or capillary network growth proximal to the residual tumor cell, and secretion of an active metalloproteinase from the residual tumor cell.

In one aspect, the present invention comprises a method of inhibiting metastases of a systemic cancer into the CNS (central nervous system) of a mammal comprising administration to the mammal of a therapeutically effective amount of a pharmaceutical composition comprising a cell-permeable fusion protein conjugate comprising a polypeptidic cell-membrane transport moiety and a Clostridium botulinum C3 exotransferase unit, or a functional analog thereof, for example a fusion protein such as BA-07.

In one aspect, a therapeutically effective amount of a pharmaceutical composition comprising a cell-permeable fusion protein conjugate comprising a polypeptidic cell-membrane transport moiety and a Clostridium botulinum C3 exotransferase unit, or a functional analog thereof, for example a fusion protein such as BA-07, can exhibit anti-angiogenic activity and is useful in the treatment of cancer.

In accordance with the present invention the active agent region of a fusion protein useful in this invention comprises an ADP-ribosyl transferase C3 region, or a functional equivalent thereof. In accordance with the present invention, a preferred ADP-ribosyl transferase C3 may be selected from the group consisting of an ADP-ribosyl transferase derived from Clostridium botulinum and a recombinant ADP-ribosyl transferase.

Alternatively, C3 can be derived from other sources such as C. limoseum or Staphylococcus aureus. C3 purified from these bacteria have enzymatic activity as of C3 from C. botulinum that is effective to ADP ribosylate Rho and cause inactivation of Rho.

In one aspect of the present invention a polypeptidic cell-membrane transport moiety can comprise a proline-rich transport domain. Examples of a proline-rich transport moiety or domain can be found in U.S. patent application Ser. No. 10/118,079, the entire disclosure of which is herein incorporated by reference in its entirety. As used herein the term “proline-rich region” refers to any linear sequence of 10 amino acids linked together by peptide amide bonds within a molecule comprising a peptide or protein, wherein at least 3 out of the 10 amino acids in the linear sequence are proline residues, wherein each proline is covalently linked in a peptide amide bond at its nitrogen and in another peptide amide bond at its carboxylic (carbonyl) site.

A proline-rich region in any 10 amino acid sequence within a peptide can comprise 2 or more proline residues and 8 or fewer non-proline amino acids.

For example, in one aspect, a proline-rich region in peptide comprising a 10 amino acid sequence within a peptide comprising 10 or more amino acids can comprise 2 proline residues and 8 non-proline amino acid residues distributed in any combination among the 10 amino acids.

In another aspect, a proline-rich region in peptide comprising a 10 amino acid sequence within a peptide comprising 10 or more amino acids can comprise 3 proline residues and 7 non-proline amino acid residues distributed in any combination among the 10 amino acids.

In another aspect, a proline-rich region in peptide comprising a 10 amino acid sequence within a peptide comprising 10 or more amino acids can comprise 4 proline residues and 6 non-proline amino acid residues distributed in any combination among the 10 amino acids.

In another aspect, a proline-rich region in peptide comprising a 10 amino acid sequence within a peptide comprising 10 or more amino acids can comprise 5 proline residues and 5 non-proline amino acid residues distributed in any combination among the 10 amino acids.

In another aspect, a proline-rich region in peptide comprising a 10 amino acid sequence within a peptide comprising 10 or more amino acids can comprise 6 proline residues and 4 non-proline amino acid residues distributed in any combination among the 10 amino acids.

In another aspect, a proline-rich region in peptide comprising a 10 amino acid sequence within a peptide comprising 10 or more amino acids can comprise 7 proline residues and 3 non-proline amino acid residues distributed in any combination among the 10 amino acids.

In another aspect, a proline-rich region in peptide comprising a 10 amino acid sequence within a peptide comprising 10 or more amino acids can comprise 8 proline residues and 2 non-proline amino acid residues distributed in any combination among the 10 amino acids.

In another aspect, a proline-rich region in peptide comprising a 10 amino acid sequence within a peptide comprising 10 or more amino acids can comprise 9 proline residues and 1 non-proline amino acid residue distributed in any combination among the 10 amino acids.

In another aspect, a proline-rich region in peptide comprising a 10 amino acid sequence within a peptide comprising 10 or more amino acids can comprise 10 proline residues.

In another aspect, a “proline-rich region” refers to an amino acid sequence region of a protein containing more prolines than that which is generally observed in naturally occurring proteins (e.g., proteins encoded by the human genome).

A “proline-rich region” of a peptide in a composition of the present invention can function to enhance the rate of transport of a fusion protein of this invention through a cell membrane.

A non-proline-rich region of a peptide or protein can comprise a sequence of 10 amino acids covalently linked by peptide bonds, which region contains zero or one proline residues.

A cell membrane transport-enhancing peptide of a composition of this invention can comprise one or more than one proline-rich region, each of which can be the same or different sequence of amino acids, and each of which is covalently linked together by a peptide bond or by the peptide bonds comprising one or more non-proline-rich amino-acid sequence which may each be the same or different when the non-proline-rich amino-acid sequence comprises more than 10 amino acids.

In another aspect of the invention, a polypeptidic cell-membrane transport moiety suitable for use in compositions and methods comprising a fusion protein of this invention can be prepared, for example, by methods modified and adapted for use in this invention as disclosed in Rojas (1998) 16: 370-375 relating to a membrane translocating sequence; in Vives (1997) 272: 16010-16017 relating to a Tat-mediated protein delivery; in Wender et al. 2000, PNAS 24: 13003-13008 related to polyargine sequences; in Derossi (1996) 271: 18188-18193 relating to antennapedia; in Canadian patent document 2,301,157 relating to conjugates containing homeodomain of antennopedia; and in U.S. Pat. Nos. 5,652,122, 5,670,617, 5,674,980, 5,747,641, and 5,804,604 relating to conjugates containing amino acids of Tat HIV protein (herein, Tat HIV protein is sometimes referred to as Tat); the entire disclosure in each of which is herein incorporated by reference in its entirety.

Several receptor-mediated transport strategies have been used to try and improve function of ADP ribosylases. These strategies or methods include fusing C2 and C3 sequences (Wilde, et al. (2001) 276: 9537-9542) and use of receptor-mediated transport with the diptheria toxin receptor (Aullo, et al. (1993) 12: 921-31). These strategies have not produced dramatically increased potency of C3 activity, unlike the activity that has been found with BA-05. Moreover, those strategies require receptor-mediated transport. This requires that the targeted cells must express a specific receptor, and must express sufficient quantities of that receptor to significantly improve transport rates. In the case of dipthera toxin, not all cells express the appropriate receptor, limiting its potential use. In contrast to these strategies, a composition of this invention comprising a polypeptide transport moiety such as, for example, BA-05 is able to cross a cell plasma membrane by a receptor-independent mechanism.

In one aspect of this invention, a preferred composition comprises a cell-permeable fusion protein conjugate comprising a proline-rich polypeptidic cell-membrane transport moiety comprising a proline-rich amino acid sequence added to the C-terminal region of a Clostridium botulinum C3 exotransferase unit, or a functional analog thereof, in a fusion protein conjugate. An especially preferred composition is a fusion protein designated BA-05. Fusion protein compositions comprising a proline-rich amino acid sequence added to the N-terminal region of a Clostridium botulinum C3 exotransferase unit, or a functional analog thereof, are sometimes referred to herein as analogs of BA-05.

In another aspect of this invention, a preferred composition comprises a cell-permeable fusion protein conjugate comprising a proline-rich polypeptidic cell-membrane transport moiety comprising a proline-rich amino acid sequence added to the N-terminal region of a Clostridium botulinum C3 exotransferase unit, or a functional analog thereof, in a fusion protein conjugate. Fusion protein compositions comprising a proline-rich amino acid sequence added to the N-terminal region of a Clostridium botulinum C3 exotransferase unit, or a functional analog thereof, are sometimes referred to herein as variants of BA-05.

The BA-05 analogs and BA-07 variants of the present invention each comprise a polypeptidic cell-membrane transport moiety and a Clostridium botulinum C3 exotransferase unit, or a functional analog thereof. Functional analogs of a Clostridium botulinum C3 exotransferase unit can comprise polypeptides such as biologically active fragments and altered-amino-acid-sequence analogs of BA-05, wherein the biological activity of such fragments and altered-amino-acid-sequence analogs of BA-05 derives from a mechanism of action essentially similar to that of BA-05. Such fragments comprise or encompass amino acid sequences having truncations of one or more amino acids relative to that in BA-05. Such fragments comprise or encompass amino acid sequences having truncations (or eliminations) of one or more amino acids relative to the sequence of amino acids in BA-05, wherein a truncation may originate from the amino or N-terminus, the carboxy or C-terminus, or from the interior of the protein sequence. Analogs and variants of BA-05 of the invention can comprise an insertion or a substitution of one or more amino acids. Compositions of this invention comprising fragments, analogs and variants useful in this invention have the biological property of BA-05 that is capable of inactivation a Rho GTPase and preferably capable of inactivation of more than one Rho GTPase.

In another aspect, compositions and methods of this invention comprise chimeric polypeptides comprising a BA-05 amino acid sequence or a truncated sequence, fused to and comprising heterologous amino acid sequences. Such heterologous sequences encompass those which, when formed into a chimera with BA-05 retain one or more biological or immunological properties of BA-05, most preferably the property of being capable of inactivation a Rho GTPase and even more preferably capable of inactivation of more than one Rho GTPase.

In another embodiment, this invention comprises a host cell transformed or transfected with nucleic acids encoding BA-05 protein or BA-07 chimeric protein. In one aspect, any host cell which produces a protein comprising a polypeptide that exhibits at least one of the biological properties of a BA-05 may be used, most preferably the property of being capable of inactivation a Rho GTPase and even more preferably capable of inactivation of more than one Rho GTPase. Representative examples of host cell types include bacterial, yeast, plant, insect, and mammalian cells. In addition, BA-05 protein or BA-05 chimeric protein may be produced in transgenic animals. Transformed or transfected host cells and transgenic animals can be obtained using materials and methods that are routinely available to one skilled in the art of molecular and cell biology. A host cell may contain a nucleic acid sequence comprising a full-length gene that encodes for BA-05 protein and which can also include a leader sequence and a C-terminal membrane anchor sequence. Alternatively, a host cell may contain a nucleic acid sequence which lacks one leader sequence or which lacks both of the leader sequences or which lacks the C-terminal membrane anchor sequence, or which lacks combinations of these sequences. In addition, nucleic acid sequences which encode a polypeptide fragment, a polypeptide variant, or a polypeptide analog, each capable of retention of the biological activity of BA-05, may also be resident in such host expression systems.

A Rho antagonist that is a recombinant protein can be made according to methods of recombinant protein technology known in the art. A protein of the present invention may be prepared from a bacterial cell extract, or through the use of recombinant techniques. BA-05 and related fusion proteins according to the invention can be produced by transformation (e.g., by transfection, by transduction, by infection) of a host cell with all or part of a BA-05-encoding DNA fragment in a suitable expression vehicle or vector. Suitable expression vehicles include: plasmids, viral particles, and phage. For insect cells, baculovirus expression vectors are suitable. The entire expression vehicle or vector, or a part thereof, can be integrated into the host cell genome by methods known in the art. In one aspect, use of an inducible expression vector is preferred.

Those skilled in the field of molecular biology will understand that any of a wide variety of expression systems can be used to provide the recombinant protein. The precise host cell used is usually not critical to the invention. For example, the BA-05 fusion protein and fusion proteins comprising functional analogs and variants and fragments of BA-05 of this invention can be produced in a prokaryotic host (e.g., E. coli or B. subtilis) or in a eukaryotic host (e.g., Saccharomyces or Pichia; mammalian cells, e.g., cells designated in the art as COS, NIH 3T3, CHO, BHK, 293, or HeLa cells; or insect cells).

To determine the relative and effective Rho antagonist activity of the compositions of this invention, a tissue culture bioassay system can be used. BA-05 at a concentration range of from about 0.01 to about 10 ug/ml is useful and is not toxic to cells.

BA-05 is stable at 37° C. for at least 24 hours. The stability of BA-05 was tested in tissue culture with the following experiment. The BA-05 was diluted in tissue culture medium, left in an incubator at 37° C. for 24 hours, then added to the bioassay system described herein, using retinal ganglion cells as the test cell type. These cells were able to extend neurites on inhibitory substrates when treated with C3 stored for 24 hours at 37 C. A minimum stability of 24 hours is achieved.

Another method to confirm that a compound is a Rho antagonist can utilize a radioactive assay to detect enzymatic activity.

Another method to detect activity can utilize a fluorescent assay to detect enzymatic activity. For example, BA-05 has at least two inherent enzymatic activities, glycohydrolase and ADP-ribosyl transferase. These enzymatic activities can act in a sequential manner to mono-ADP-ribosylate and inactivate the GTP-binding protein RhoA by trapping ADP-ribosylated Rho in a complex with guanine-nucleotide dissociation inhibitor-1 (GDI-1). In the first step of the reaction, the glycohydrolase activity hydrolyses the N-glycosidic bond between nicotinamide and adenine dinucleotide phosphate-ribose (ADP-ribose) in the nicotinamide adenine dinucleotide (NAD⁺) molecule. The second step, catalysed by the ADP-ribosyltransferase, results in the formation of ADP-ribose-RhoA. The enzyme assays can measure the glycohydrolase activity of a fusion protein of this invention such as BA-05 and BA-07 by following the formation of ADP-ribose.

In one aspect, the present invention comprises a pharmaceutical composition useful for suppressing malignant transformation and metastasis, the pharmaceutical composition comprising a pharmaceutically acceptable diluent or carrier and a therapeutically effective amount of composition of this invention, preferably a fusion protein of this invention.

In one embodiment, a composition of this invention can comprise an active member selected from the group consisting of a drug delivery construct as described herein, a drug conjugate as described herein, and a fusion protein as described herein (e.g. including pharmaceutically acceptable chemical equivalents thereof).

Formulation of BA-05 and Other Compositions of this Invention

Compositions and methods of this invention can comprise a pharmaceutically acceptable carrier and a therapeutically effective amount of a pharmaceutical composition comprising a cell-permeable fusion protein conjugate comprising a polypeptidic cell-membrane transport moiety and a Clostridium botulinum C3 exotransferase unit, or a functional analog thereof. In one aspect, a wide variety of polymeric carriers may be utilized in a formulation of this invention. Representative examples of polymeric carriers include poly(ethylene-co-vinyl acetate) (PVA) and partially hydrolyzed poly(ethylene-co-vinyl acetate) as poly(ethylene-co-vinyl acetate-co-vinyl alcohol), any of which can be optionally crosslinked up to about 40% cross-linked; poly-D,L-lactic acid including low molecular weight oligomers and high molecular weight polymers thereof; poly-L-lactic acid including low molecular weight oligomers and high molecular weight polymers thereof; polyglycolic acid (PGA); copolymers of lactic acid and glycolic acid; polycaprolactone; polyvalerolactone; poly (anhydrides), copolymers of polycaprolactone with polyethylene glycol; copolymers of polylactic acid with polyethylene glycol, polyethylene glycol; and combinations and blends thereof. Copolymers can comprise from about 1% to about 99% by weight of a monomer unit. Blends of a first polymer and a second polymer can comprise from about 1% to about 99% by weight of the first polymer and from about 99% to about 1% of the second polymer.

### Application of BA-05 to Arrest Tumor Spread

Compositions of the present invention, such as anti-neoplastic and anti-metastatic compositions, may be formulated in a variety of forms. For example, in one embodiment, a pharmaceutical composition comprising a therapeutically effective amount of a cell-permeable fusion protein conjugate comprising a polypeptidic cell-membrane transport moiety and a Clostridium botulinum C3 exotransferase unit, or a functional analog thereof, can comprise a microsphere, wherein the fusion protein is blended with or embibed into a matrix comprising a pharmaceutically acceptable polymeric carrier, optionally in the presence of water (from about 0.1% to about 15% in one embodiment; alternatively, the microsphere suspended in a aqueous medium in another embodiment), a pharmaceutically acceptable buffer salt, a pharmaceutically acceptable surface active agent, a pharmaceutically acceptable carbohydrate, a pharmaceutically acceptable emollient, and the like.

In another embodiment, a pharmaceutical composition comprising a therapeutically effective amount of a cell-permeable fusion protein conjugate comprising a polypeptidic cell-membrane transport moiety and a Clostridium botulinum C3 exotransferase unit, or a functional analog thereof, can comprise a paste, a cream, an ointment, a suppository, a suspension in a pharmaceutically acceptable oil, and the like.

In another embodiment, a pharmaceutical composition comprising a therapeutically effective amount of a cell-permeable fusion protein conjugate comprising a polypeptidic cell-membrane transport moiety and a Clostridium botulinum C3 exotransferase unit, or a functional analog thereof, can comprise a film, for example wherein the fusion protein is blended or mixed together with a pharmaceutically acceptable carrier such as an aqueous gelatin or an aqueous protein or a polymeric carrier or a combination thereof, optionally in the presence of a cross-linking agent species which can crosslink the carrier, the blend then coated into a film or laminate, optionally in the present of a film base or a support or matrix, and dried or dehydrated, optionally by the addition of heat or by lyophilization. Films can be prepared in unit dosage forms or in bulk and divided and cut into unit dosage forms.

In another embodiment, a pharmaceutical composition comprising a therapeutically effective amount of a cell-permeable fusion protein conjugate comprising a polypeptidic cell-membrane transport moiety and a Clostridium botulinum C3 exotransferase unit, or a functional analog thereof, can comprise an aerosol or sprayable or aerosolizable composition such as a suspension or solution of the fusion protein in a pharmaceutically acceptable fluid such as an aqueous solution of a buffer, optionally with a tonicity modifier; in a pharmaceutically acceptable fluid such as a supercritical or liquefied gas such as carbon dioxide or propane or a low molecular weight fluorocarbon or fluorohydrocarbon or bromofluorocarbon or chlorofluorocarbon and the like, each of which is a gas at 37° C. and ambient pressure, the composition suitable for use, for example, in inhalation or as an aerosol such as a spray-on-a-tissue-surface application.

In another aspect, the compositions of the present invention may be formulated to contain a fusion protein such as BA-05 and an additional anti-neoplastic and anti-metastatic factor or agent.

In another aspect, the compositions of the present invention may be formulated to contain a variety of additional compounds, in order to provide the formulated fusion protein formulations with certain physical properties (e.g., elasticity related to incorporation of a pharmaceutically acceptable plasticizing agent, a particular melting point such as about 30° C. such as by use of a polyethylene glycol, or a specified release rate which may be related to degree of crosslinking or rate of hydration in a matrix or to solubilization of a matrix, or to preferential solubilization of one component of a matrix which can leave pores in the matrix through which a carrier fluid such a water can assist in transport of the fusion protein out of the matrix and into or onto a desired site in the body of a mammal.

Within certain embodiments of the invention, compositions may be combined in order to achieve a desired effect (e.g., two or more compositions of microspheres of this invention may be combined in order to achieve a modified net release rate of a fusion protein of this invention such as both a quick and a slow or prolonged release of one or more anti-neoplastic and anti-metastatic factor).

Compositions of the present invention such as those comprising BA-05 may be administered either alone, or in combination with a pharmaceutically acceptable carrier, and/or pharmaceutically and physiologically compatible excipients, diluents, tonicity modifying agents, buffers, and the like. Preferably, such carriers are acceptably nontoxic to a recipient when used in combination with the dosages and at the therapeutically effective concentrations of the fusion protein employed.

In one aspect, preparation of a pharmaceutical composition of this invention comprises combining the therapeutically effective amount of a fusion protein of this invention with one or more components of a carrier such as water; a pharmaceutically acceptable buffer salt or buffer solution; a pharmaceutically acceptable antioxidant such as ascorbic acid; one or more low molecular weight pharmaceutically acceptable polypeptide (e.g., a peptide comprising about 2 to about 10 amino acid residues); one or more pharmaceutically acceptable protein; one or more pharmaceutically acceptable amino acid such as an essential-to-human amino acid; one or more pharmaceutically acceptable carbohydrate or carbohydrate-derived material such as glucose, sucrose, sorbitol, trehalose, mannitol, maltodextrin, dextrins, cyclodextrin, and combinations thereof, in one aspect such carbohydrate preferably comprising a non-reducing carbohydrate such as a non-reducing sugar when avoidance of the Maillard reaction (which takes place when components such as a reducing sugar and an amino acid or peptide or protein react together) is desired, or in another aspect such carbohydrate preferably comprising a reducing carbohydrate such a reducing sugar when a Maillard reaction is desired; a pharmaceutically acceptable chelating agent such as EDTA, or DTPA, which is a chelating agent for a metal ion such a divalent metal ion (e.g., Ca+2, Fe+2 and the like) or a trivalent metal ion (e.g., Fe+3, Y+3, Ln+3, Eu+3 and other lanthanides, and the like, and which may optionally comprise a radionuclide); glutathione; and other stabilizers and excipients known in the are of formulation of a protein material. Preferred carriers comprise sterile buffered saline at a pH in the range from about 6 to about 8, preferably at about pH 7.4, and a sterile isotonic composition comprising saline mixed with pharmaceutically acceptable nonspecific serum albumin.

The pharmaceutical compositions of this invention can be sterile, sterilizable, and sterilized. A preferred method of sterilization comprises filtration of a pharmaceutical composition through a 0.2 micron filter in a sterile environment. The sterile filtered composition can be filled in a vial, preferably into a sterile vial, in a unit dosage volume amount or in an integral multiple of a unit dosage amount (e.g., as 2 unit dosage amount, 3 unit dosage amounts, 4 unit dosage amounts, et cetera), preferably under an inert atmosphere such as sterile nitrogen or argon, and the vials sealed with a pharmaceutically acceptable stopper, optionally with a crimp cap. In another aspect, pharmaceutical composition is dried by removal of water, for example the aqueous medium can be removed from each vial by a drying process such as by lyophilization or evaporation to leave a dried or dehydrated matrix comprising the fusion protein of this invention, before sealing and capping of the vial. In another aspect, the carrier can comprise a sterile or sterilizable hypertonic solution of a pharmaceutically acceptable matrix-forming material or excipient that is compatible with the fusion protein, for example, such as a pharmaceutically acceptable non-reducing carbohydrate, together with a compound or fusion protein of the invention, which hypertonic solution can be placed in a vial and dried (e.g., by lyophilization) to provide a matrix comprising the fusion protein and the matrix-forming excipient, which can be sealed in the vial with a cap. Prior to use, sterile water can be added to the vial, for example vial sterile syringe or cannula, which water will dissolve the matrix to provide a solution or suspension of the fusion protein. Sufficient water can be added to provide the reconstituted solution or suspension as an isotonic solution suitable for injectable or implantable use.

Pharmaceutical compositions of this invention may be prepared to be suitable for administration to a mammal, such as a patient in need of treatment, by a variety of different routes. Preferred routes of administration include for example intrarticular, intraocular, intranasal, intraneural, intradermal, intraosteal, sublingual, oral, topical, intravesical, intrathecal, intravenous, intraperitoneal, intracranial, intramuscular, subcutaneous, inhalation or atomization and inhalation, or application directly into a tumor or disease site or on or into the margins remaining after resection of a tumor. Other representative routes of administration comprise enteral optionally together with a gastroscopic procedure, and colonoscopy, each of which do can be outpatient procedures and not require full operating room procedures and prolonged hospitalization, but may require the presence of medical personnel.

The pharmaceutical compositions provided herein may be placed within containers along with packaging material which provides instructions regarding the use of such materials. Generally, such instructions will include a description of the concentration of the active agent, as well as within certain embodiments, relative amounts or identities of excipient ingredients or diluents (e.g., water, saline or PBS). In addition, it may be necessary to reconstitute the anti-neoplastic and anti-metastatic composition, or pharmaceutical composition to a pharmaceutically acceptable solution or suspension by the addition of water and optionally also with shaking or sonication.

The pharmaceutical compositions of this invention may be utilized in a wide variety of surgical procedures. For example, within one aspect of the present invention a pharmaceutical composition (in the form of, for example, a solution or suspension or powder suitable from application in an atomized or aerosol or spray form, or coated in a film) may be applied by spraying (a sprayable or aerosol-forming form) or by lamination (of a film) onto a surface of an area of tissue in a patient in need of treatment prior to, during, or after a surgical removal of a tumor such as a first tumor, and optionally an amount of normal tissue immediately proximal to the tumor, from the area of tissue, which removal leaving a margin of normal tissue around the excision site of the tumor (tumor margin) in the area of tissue. In one aspect, this procedure can prevent or substantially retard or inhibit metastatic growth of a second tumor in the normal surrounding tissues after removal of the first tumor in the patient. In another aspect, this procedure can prevent the spread of disease (e.g., cancer) to surrounding tissues. Within other aspects of the present invention, a pharmaceutical composition of the present invention (e.g., in the form of a spray or an aerosol) may be delivered via an endoscopic procedure, wherein the composition is sprayed or aerosolized inside a patient to provide a coating comprising a fusion protein of this invention on a tumor and/or tissue surrounding and proximal to a tumor inside a patient, which tumor is accessed or visualized by endoscopic means. In another aspect, coating of a pharmaceutical composition on to a tissue proximal to a tumor or proximal to the site of excision of a tumor can inhibit angiogenesis in the region of tissue that is coated by the pharmaceutical composition.

Within yet other aspects of the present invention, a pharmaceutical composition of this invention can be coated onto the surface of an implantable device such as a surgical mesh, wire, stent, prosthetic device, and the like, to form a coated device, the coating comprising a fusion protein of this invention and optionally a polymeric carrier, which coated device may be implanted in a tissue or organ in a patient as part of a surgical treatment, such as a surgical removal of a cancerous or benign tumor, which pharmaceutical composition can prevent or inhibit or delay or retard growth of a second tumor proximal to the location of the device, and in another aspect, can also prevent or inhibit or delay or retard growth of a second tumor in a tissue or organ remote from the site of the implanted device. The concentration of the fusion protein can be from 0.01% to about 20% by weight of the carrier that forms a coating on the device, and the thickness of the coating can be from about 20 micrometers to about 1 millimeter. The coating can be applied by coating means known in the art of coating devices. For example, a coating comprising a pharmaceutical composition of this invention can be applied to the surface of a device by means of a spray or aerosol applicator in which the pharmaceutical composition as a solution in a liquid or fluid comprising a solvent or as a suspension in a liquid or fluid, which liquid or fluid can evaporate during and after application as a spray or an aerosol, is sprayed or aerosolized onto the surface of a device. Optionally, the coated composition can comprise reactive chemical functional groups such as olefins or anhydride groups or active esters or Michael reaction acceptors such as a carbon-carbon double bond conjugated to a carbonyl group, which double bond can react with an amine of a protein or peptide or gelatin such as a carrier protein, which reactive chemical functional groups can chemically or photochemically form crosslinks in the carrier, which can prevent solubilization or limit or modify or control swelling (as a function of concentration of the reactive functional groups or the time of exposure to crosslinking conditions such as ultraviolet or gamma irradiation of the coated device) of the coated carrier by aqueous fluid in the tissue in which the device is implanted. Control of swelling can be useful to control the rate at which the fusion protein of this invention migrates from the device into the tissue proximal to the device and further into the body of the patient. A wide variety of crosslinking chemistry known in the art can be useful in this aspect of the invention as long as the biological activity of the fusion protein is not negated or eliminated. If an organic solvent or supercritical fluid or liquefied gas is used in the coating process, then a pharmaceutically acceptable carrier can be selected which does not immediately dissolve in the aqueous medium present in tissue proximal to the site of implantation but permits permeation of the fusion protein into the aqueous medium.

Other methods of coating can be used such as dip coating of a composition, painting, curtain coating, and lamination of a pharmaceutical composition of this invention.

In one embodiment, the surface of a device can be first coated with a first coating layer or primer layer which is then subsequently coated with a pharmaceutical composition of this invention as a second coating layer. The primer layer can be selected to adhere to the surface of the metal or polymeric device and to adhere to the carrier of the second coating layer. The primer layer can also comprise immobilized chemical functional groups (e.g., which can be attached to a polymer in the primer layer) and which can form crosslinking bonds with the second layer. The primer layer can optionally contain relatively mobile molecules comprising for example two or more reactive functional groups, which molecules can migrate into the second layer and react with chemical functional groups therein to form crosslinking molecular bridges.

In an other embodiment, a pharmaceutically acceptable third layer can be overcoated on the second layer, the third layer optionally void of fusion protein. The third layer can serve to control or modify the release rate of the fusion protein from the device, for example by being able to dissolve or swell or increase its permeability with respect to water or the fusion protein as a function of time to expose the second layer comprising the pharmaceutical composition of this invention to aqueous media from the tissue.

Within one embodiment of the invention a surgical mesh device comprising a pharmaceutical composition of the present invention coated on the surface of a wire or polymer mesh may be utilized or implanted in a patient such as during an abdominal cancer resection surgical procedure on the patient (e.g., subsequent to colon resection) in order to provide support to the residual tissue structure. The coated mesh device can release a therapeutically effective amount of the active component (such as BA-07) of the pharmaceutical composition sufficient to prevent reoccurrence of the cancer by prevention of growth of a second tumor proximal to the site of implantation of the coated device. The fusion protein can migrate from the device at a rate sufficient to provide a therapeutically effective concentration range in the tissue proximal to the device.

A currently preferred concentration range is about 0.0001 micrograms of fusion protein per cubic centimeter (cc) of tissue to about 100 micrograms per cubic centimeter of tissue can be useful. A currently more preferred therapeutically effective concentration range is about 0.001 micrograms per cc to about 50 micrograms per cc of tissue.

In another embodiment a coated mesh device can release a therapeutically effective amount of the active component (such as BA-07) of the pharmaceutical composition sufficient to prevent reoccurrence of the cancer by prevention of growth of a second tumor remote from the site of implantation of the coated device.

Within further aspects of the present invention, methods are provided for treatment of a patient at the site of residual tissue left at the margin of excision of a first tumor (a tumor excision site) comprising administration of a pharmaceutical composition of this invention to a residual tissue at a resection margin of a first tumor of a cancer subsequent to excision of the first tumor, such that recurrence of a second tumor of the cancer and formation of new blood vessels at the site of residual tissue at the first tumor margin is inhibited. Within one embodiment of the invention, a pharmaceutical composition of the invention such as a pharmaceutical composition comprising BA-07 is administered directly to the residual tissue at a tumor excision site (e.g., applied by swabbing, brushing, painting, spraying, aerosolization, injection, lavage, soaking, or otherwise coating the resection margins of the tumor with the pharmaceutical composition. Alternatively, a pharmaceutical composition of this invention such as a pharmaceutical composition comprising BA-07 in the form of a surgical paste, ointment, cream, suspension, gel, and the like can be applied to the surface of the tissue.

In a preferred embodiment of the invention, a pharmaceutical composition of this invention comprising a fusion protein such as BA-07 is applied to residual tissue at the site of excision of a tumor of the liver such as after a hepatic resection for malignancy.

In another preferred embodiment of the invention, a pharmaceutical composition of this invention comprising a fusion protein such as BA-07 is applied after a neurosurgical operation (e.g., related to removal of a tumor of the brain).

Within one aspect of the present invention, a pharmaceutical composition of this invention comprising a fusion protein such as BA-07 may be administered to a tumor resection margin residual tissue of a wide variety of tumors, including for example, breast, colon, brain and hepatic tumors. For example, within one embodiment of the invention, a pharmaceutical composition of this invention comprising a fusion protein such as BA-05 may be administered to the residual tissue proximal to the site of removal of a first tumor of neurological cancer subsequent to excision of the first tumor, such that spread of cells of the cancer into the residual tissue and formation of a second tumor and formation of new blood vessels in the tissue at the residual margin site of the first tumor are inhibited.

The brain is highly functionally localized: i.e., each specific anatomical region is specialized to carry out a specific function. The location of a cancer in the brain of a patient (and brain pathology) can be more important than the type of tissue or tumor type. A relatively small tumor or lesion in a key area of the brain can be far more devastating than a much larger lesion in a relatively less important area of the brain. A lesion on the surface of the brain may be relatively easy to resect surgically, while a tumor of comparable size but located deep in the brain may not be relatively easy to resect surgically because access to the deep tumor could require disruption of intervening tissue such as by cutting through many vital structures to reach or access and remove the deep tumor. In addition, benign tumors in the brain can be dangerous to a patient. A benign tumor may grow in a key area and cause significant damage to surrounding brain tissue and function. Although a benign tumor can be cured by surgical resection, removal of the tumor from deep tissue may not be possible. If left unchecked a benign tumor can grow, increase in volume, and cause increased intracranial pressure If such a condition is left untreated, vital structures in the brain can be compressed, and death of the patient can result. The incidence of CNS (central nervous system) malignancies is about 8 to 16 cases per 100,000 people. The prognosis of a primary malignancy of the brain is dismal, with a median survival of less than one year, even following surgical resection. Brain tumors, especially gliomas, are predominantly a local disease which can recur within about 2 centimeters of the original focus of disease after surgical removal.

Representative examples of brain tumors which may be treated utilizing the compositions and methods described herein include glial tumors such as anaplastic astrocytoma, glioblastoma multiform, pilocytic astrocytoma, oligodendroglioma, ependymoma, myxopapillary ependymoma, subependymoma, choroid plexus papilloma; neuron tumors such as neuroblastoma, ganglioneuroblastoma, ganglioneuroma, and medulloblastoma; pineal gland tumors such as pineoblastoma and pineocytoma; menigeal tumors such as meningioma, meningeal hemangiopericytoma, meningeal sarcoma; tumors of nerve sheath cells such as Schwannoma (neurolemmoma) and neurofibroma; lymphomas such as Hodgkin's lymphoma and non-Hodgkin's lymphoma, primary and secondary subtypes of Hodgkin's lymphoma, primary and secondary subtypes of non-Hodgkin's lymphoma (and including numerous subtypes of these, both primary and secondary); malformative tumors such as craniopharyngioma, epidermoid cysts, dermoid cysts and colloid cysts; and metastatic tumors located in the brain which can be derived from virtually any tumor, the most common being derived from tumors of the lung, breast, melanoma, kidney, and gastrointestinal tract.

In one embodiment of this invention, the pharmaceutical compositions of the invention may be applied locally, such as topically or by topical application, in a unit dosage amount. Such administration can comprise application of a pharmaceutical composition to the external portion of the epidermis, topical administration to tissue exposed to topical administration in the mouth cavity, and the topical instillation onto exposed tissue in the eye, ear and nose, such that no more than about 10% and preferably no more than 1% of the unit dose of a fusion protein of this invention (such as BA-07) enters the blood stream of a patient directly.

In another embodiment of this invention, the pharmaceutical compositions of the invention may be administered systemically such as by injection into a blood vessel or lymph vessel, for example by intravenous injection.

Additional modes of administration include intraperitoneal, subcutaneous, intramuscular, rectal (e.g, as a suppository dosage form), vaginal (e.g., as a pessary), and peroral delivery. Dosage forms of this invention can act as a depot comprising a fusion protein of this invention, which fusion protein can migrate into tissue proximal to the site of the depot.

Compositions for use in topical administration include, e.g., liquid or gel preparations preferably suitable for penetration through the skin such as creams, liniments (e.g., applied to the skin by friction), lotions, oils, ointments, pastes, and drops suitable for delivery to tissue of organs such as the eye, ear, nose.

In one embodiment of the invention, the fusion protein can have molecular weight of from about 240,000 daltons to about 300,000 daltons.

In another embodiment, the compositions provided herein may be formed into films with a thickness of between 100 micrometers and 2 millimeters, or thermologically active compositions which are liquid at one temperature (e.g., above about 25° C.) and solid or semi-solid (e.g., below about 25° C.).

Within another aspect of the present invention, methods are provided for treating residual tissue remaining at a malignant tumor excision site, comprising administering a pharmaceutical composition of this invention comprising a fusion protein such as BA-05 to the residual resection margins of a tumor in a patient subsequent to excision of the tumor from the patient, such that the local recurrence of cancer and the formation of new blood vessels at the site is inhibited.

Within another aspect of the present invention, methods are provided for treating a tumor excision site, comprising administering a composition comprising BA-05 to the resection margin of a tumor subsequent to excision, such that the local recurrence of cancer and the formation of new blood vessels at the site is inhibited.

Another aspect of the invention comprises a pharmaceutical composition of this invention in a kit of parts such as a kit comprising a container and a pharmaceutical composition of this invention; a kit comprising a sealed vial and a pharmaceutical composition of this invention; a kit comprising a sterile syringe and a pharmaceutical composition of this invention; a kit comprising a sterile syringe containing a pharmaceutical composition of this invention; a kit comprising a spray or aerosol applicator and a pharmaceutical composition of this invention; a kit comprising a brush applicator and a pharmaceutical composition of this invention; a kit comprising a cannula and a pharmaceutical composition of this invention; a kit comprising a powder applicator and a pharmaceutical composition of this invention (which powder applicator can be used to administer a pharmaceutical dosage form of this invention as a powder by sprinkle application of a dried (e.g., lyophilized) powder in a topical application to a tissue; a kit comprising a coated implantable device and a pharmaceutical composition of this invention, wherein administration is by implantation.

Pharmaceutical products are provided, comprising for example, a fusion protein such as BA-05 which disrupts Rho signaling, in a container; and device such as a syringe or tool or brush or applicator device (such as a spray or aerosol applicator device in a second container, to be used for applying the fusion protein such as BA-05 to the tissue forming the walls of a tumor cavity after surgical removal of the tumor, or applying to the skin, for example after removal of a malignant melanoma.

These and other aspects of the present invention will become evident upon reference to the associated detailed description and attached figures.

### BRIEF DESCRIPTION OF THE FIGURES

FIG. 1 illustrates the effect of a composition of this invention comprising a fusion protein, BA-07, on the proliferation of HEC1B human endometrial adenocarcinoma cells as measured by tritiated thymidine incorporation. The vehicle (10) is phosphate buffered saline, and BA-07 is used at concentrations of 1 μg/ml (11), 10 μg/ml (12) and 50 μg/ml (13). Cancer cell proliferation is reduced in a dose dependent manner.

FIG. 2 illustrates the effect of a composition of this invention comprising a fusion protein, BA-07, on the proliferation of SK-MEL-1 human melanoma cells as measured by tritiated thymidine incorporation. The vehicle is phosphate buffered saline, and BA-07 is used at concentrations of 1 μg/ml, 10 μg/ml, and 50 μg/ml. Cancer cell proliferation is reduced in a dose dependent manner.

FIG. 3A illustrates tube formation formation by HUVEC endothelial cells cultured in a Matrigel™ matrix. This assay is a cell culture assay for antiogenesis. Tube formation can be seen in the control which does not contain a fusion protein of this invention, FIG. 3A (box 30).

FIG. 3B illustrates a reduction in tube formation of HUVEC endothelial cells cultured in a Matrigel™ matrix. Cultures treated with a composition of this invention comprising a fusion protein, BA-07, had fewer tubes demonstrating an inhibition of angiogenesis, as shown in FIG. 3B, box 31.

FIG. 4 shows the inhibition of growth of TK-10 human renal carcinoma cells by a composition of this invention comprising a fusion protein, BA-07, as measured by a sulforhodamine B (SRB) growth inhibition assay. The fusion protein, BA-07, is used at concentrations of 0.1 μg/ml, 1 μg/ml, 10 μg/ml, and 100 pg/ml. At all concentrations used, cancer cell proliferation is reduced. Reduction in cancer cell proliferation is dose dependent. At a concentration of fusion protein of 100 μg/ml, the composition of the invention induced cell death of cancer cells.

FIG. 5 shows the inhibition of growth of HOP-62 Non-small cell lung cancer cells by a composition of this invention comprising a fusion protein, BA-07, as measure by a sulforhodamine B (SRB) growth inhibition assay. The fusion protein, BA-07, is used at concentrations of 0.1 μg/ml, 1 μg/ml, 10 μg/ml, and 100 μg/ml. At all concentrations used, cancer cell proliferation is reduced. Reduction of cancer cell proliferation is dose dependent.

FIG. 6 shows the inhibition of growth of SF-286 CNS cancer cells by a composition of this invention comprising a fusion protein, BA-07, as measured by a sulforhodamine B (SRB) growth inhibition assay. The fusion protein, BA-07, is used at concentrations of 0.1 μg/ml, 1 μg/ml, 10 μg/ml, and 100 pg/ml. At all concentrations used, cancer cell proliferation is reduced. Reduction of cancer cell proliferation is dose dependent.

FIG. 7 shows reduction in levels of activated RhoA after incubation with 10 micrograms per milliliter of fusion protein at 1 hour, 2 hours, 4 hours, 6 hours, and 24 hours after administration of a pharmaceutical composition comprising a fusion protein of this invention and a pharmaceutically acceptable vehicle.

FIG. 8 shows the inhibition of growth (as % growth versus a vehicle control as reference) of Caki-1 renal carcinoma cells by a composition comprising a fusion proteins as BA-07, the % growth measured with an SRB assay at relative concentrations of fusion protein of 0.1, 1, 10, and 100.

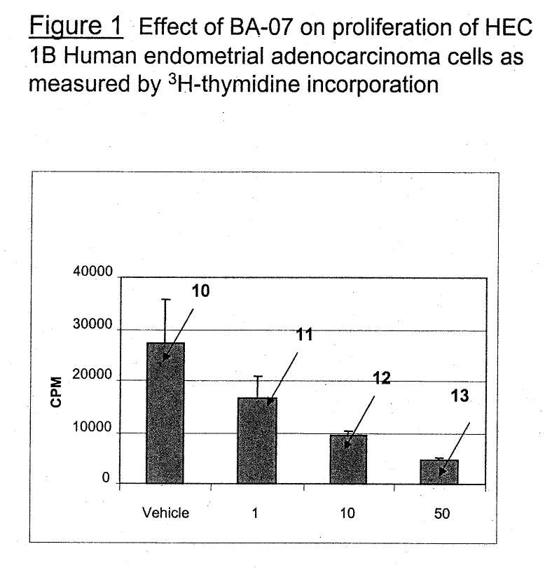

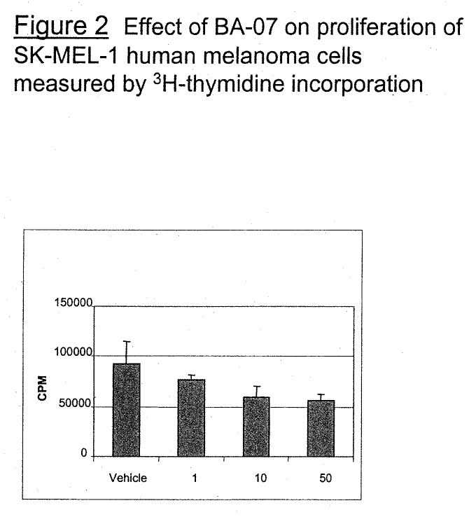

### DETAILED DESCRIPTION

All references set forth herein which describe in more detail procedures, devices or compositions relevant to this invention are incorporated by reference in their entirety.

A Method for Making a Fusion Protein of this Invention Such as BA-05

BA-05 is the name given to the protein of this invention made by ligating a cDNA sequence encoding C3 to a fusogenic 19-mer peptide. To demonstrate the method for making a fusion protein of this invention, an example of an antennapedia sequence added to the C-terminus of the C3 polypeptide can be used.

The stop codon at the 3′ end of the DNA sequence can be replaced with an EcoR1 site by polymerase chain reaction (PCR) using the primers 5′GAA TTC TTT AGG ATT GAT AGC TGT GCC 3′ (SEQ ID NO: 1) and 5′GGT GGC GAC CAT CCT CCA AAA 3′ (SEQ ID NO: 2). The PCR product can be sub-cloned into a pSTBlue-1 vector (Novagen, city), then cloned into a pGEX-4T vector using BamH I and Not I restriction site. This vector can be called pGEX-4T/C3. An antennapedia sequence useful to add to the 3′ end of C3 in pGEX-4T/C3 can be created by PCR from the pET-3a vector (Bloch-Gallego (1993) 120: 485-492; and Derossi (1994) 269: 10444-10450), subcloned into a pSTBlue-1 blunt vector, then cloned into the pGEX-4T/C3, using the restriction sites EcoR I and Sal I, creating pGEX-4T/C3APL.

DNA sequence analysis can be performed on the sequence producing the best response according to this invention.

pGEX-4T/C3APL clone (Seq ID NO: 3) is a currently preferred sequence and provides a protein that is a preferred composition of this invention.

An example of a C3-like fusion protein is denoted pGEX-4T/C3APLT (Seq ID NO: 4).

Two PCR primers are designed to transfer one series of recombinant constructs (BA-05) into the pET system: Upper primer: 5′ ggatctggttccgcgtcatatgtctagagtcgacctg 3′ (Seq ID NO: 5) Lower primer: 5′ cgcggatccattagttctccttcttccacttc 3′ (SEQ ID NO: 6).

A BamHI site at the 5′ end of Seq ID NO: 6 is ggatccatta; the TGA is replaced by TAAT (atta, in SEQ ID NO: 6).

A program useful to amplify the product using Pfu polymerase comprises: 95° C. 5′ 1 cycle, then 94° C. 2′→56° C. 2′→70° C. 2′ 10 cycles, then 94° C. 2′→70° C. 3′ 30 cycles and hold at 4° C. A QIAEXII kit (Qiagen) can be used to purify an agarose gel slice containing a desired DNA band. The insert and vector are digested with BamHI and NdeI following the instructions of the manufacturer, purified using agarose gel electrophoresis and a QIAEXII kit (Qiagen), and incubated together overnight with T4 DNA ligase following the manufacturer's directions.

E. Coli (DH5alpha, or preferably, XL1-Blue) is transformed with the ligation mixture. The clones can be checked by small scale induction and SDS-PAGE and can be assured by immunoblotting of the crude lysates with anti-C3 antibody. Plasmid DNA is purified, and can be assessed for purity. DNA sequencing can be performed (e.g., by LiCor technology in which the entire strand is sequenced for the full length of the clone).

A first construct prepared in this fashion (pET3a-BA-07, SEQ ID NO:7) matched the theoretical DNA sequence of construct pGEX/APLT with a slight change in the 5.

A second construct, pET9a-BA-07, can be prepared by subcloning the insert from pET3a-BA-07 into the pET9a vector by cleaving the pET3a construct with BamHI and NdeI (New England BioLabs, Beverly, Mass.) according to the manufacturers instructions. pET9a plasmid DNA can be cleaved with the same enzymes. The insert DNA and the vector DNA can be purified by agarose gel electrophoresis. The insert can be ligated into the new vector using T4 DNA ligase (New England BioLabs, Beverly, Mass.). The ligated DNA can be transformed into DH5alpha cells and DNA can be prepared using QIAGEN mini and maxi kits. Clones can be characterized by restriction digestion and DNA sequencing of the insert in both directions (e.g., BioS&T, Lachine, Quebec). The construct DNA can be transformed into BL21 (DE3) cells and BL21(DE3)/pLysS cells.

pET9a-BA-07 protein expression (SEQ ID NO: 8) is superior in BL21(DE3) compared to BL21(DE3)/pLysS.

The proteins of the present invention may be prepared from bacterial cell extracts, or through the use of recombinant techniques by transformation, transfection, or infection of a host cell with all or part of a fusion protein-encoding DNA fragment such as a BA-05-encoding DNA fragment) with an antennapedia-derived transport sequence in a suitable expression vehicle.

One skilled in the field of molecular biology will understand that any of a wide variety of expression systems can be used to provide a recombinant protein of this invention. The precise host cell used is not usually critical to the invention, but variations in yields are expected from one host cell type to another.

A fusion protein can be purified by utilising protein purification techniques known in the art such as affinity purification techniques or column chromatography using resins that separate molecules on the basis of properties such as charge, size and hydrophobicity. Useful affinity techniques include those employing an antibody (e.g., GST) specific for the fusion protein being expressed. Histidine-tagged proteins can be selectively eluted with imidazole-containing buffers. Alternatively, recombinant protein can be fused to an immunoglobulin Fc domain. Such a fusion protein can be readily purified using a protein A column.

Any of these techniques can be automated and optimized to provide superior reproducibility and high throughput by use of commercially available liquid chromatography equipment specialized for protein purification. It is envisioned that small molecule, peptide or other mimetics of the above described antagonists are also encompassed by the invention.

Bioactivity Evaluations of a Pharmaceutical Composition Comprising a Fusion Protein of this Invention Such as BA-05

### Change in Rho Inactivation

The ability of BA-05 and BA-07 to inactivate Rho can be demonstrated using a cell culture assay. In this assay the cancer cell line is plated on tissue culture under the conditions that are to be utilized. For example, NG108 cells can be plated and left to proliferate until semi-confluent. NG108 is a neuroblastoma X glioma formed by Sendai virus-induced fusion of the mouse neuroblastoma clone N18TG-2 and the rat glioma clone C6 BU-1. The cells are then harvested, homogenized, and a Rho pull down assay is performed. The pull down assay uses a “bait” that binds to active Rho. In our assay we can use, for example, Rho binding domain (RBD) from Rhoteckin. Other proteins, such as Rho kinase, can also be used. The “bait” is linked to a bead so that it can be precipitated from the homogenate. RBD binds to the GTP-Rho in the homogenate and does not bind to GDP-Rho. In this way, active Rho in the cell culture can be assessed quantitatively. The extent that BA-07 inactivates Rho in a cell line can be demonstrated by treating a sample of cells of the cell line before performing a pull down assay.

A pull-down assay can be used to determine the amount of active Rho in a solid tumour. A tumour sample is homogenized in buffer, a pull-down assay performed, and the amount of GTP Rho can be compared with the amount found in a non-cancerous tissue. This assay to detect active Rho can be used as a diagnostic for tumours that comprise cells with highly activated levels of Rho and which can respond according to this invention, for example to BA-07 therapy. Measure of activated Rho can be more sensitive than simply examining expression levels of Rho.

An in situ pull down assay can be used to detect GTP-Rho in histological sections. For this assay, cryosections (each about 16 μm in thickness) of tumour samples are incubated, after post fixation with 4% PFA, with a bacterial lysate containing the RBG-GST overnight at 4° C. The sections are then washed 3 times in TBS, blocked in 3% BSA for about 1 hr at room temperature and incubated with an anti-GST antibody (Cell signalling, New England Biolabs, Mississauga, Canada) and with cell-type specific antibodies to identify specific cells, and incubated overnight at 4° C. the sections are washed in TBS and incubated for 2 hr at room temperature with FITC, Texas Red or Rhodamine conjugated secondary antibodies to reveal immunoreactivity (Jackson ImmunoResearch, Mississauga, Canada).

### DNA and Protein Sequence Details of BA-05

With respect to this invention, a useful fusion protein designated as BA-05 has the following DNA coding sequence (SEQ ID No.: 9) here displayed using conventional G, A, T, and C nomenclature. In oligonucleotide sequences of this invention, the symbols G, C, A, and T have their conventional meaning.

| | |
|---|---|
| GGATCCTCTA GAGTCGACCT GCAGGCATGC AATGCTTATT CCATTAATCA | 50 |
| AAAGGCTTAT TCAAATACTT ACCAGGAGTT TACTAATATT GATCAAGCAA | 100 |
| AAGCTTGGGG TAATGCTCAG TATAAAAAGT ATGGACTAAG CAAATCAGAA | 150 |
| AAAGAAGCTA TAGTATCATA TACTAAAAGC GCTAGTGAAA TAAATGGAAA | 200 |
| GCTAAGACAA AATAAGGGAG TTATCAATGG ATTTCCTTCA AATTTAATAA | 250 |
| AACAAGTTGA ACTTTTAGAT AAATCTTTTA ATAAAATGAA GACCCCTGAA | 300 |
| AATATTATGT TATTTAGAGG CGACGACCCT GCTTATTTAG GAACAGAATT | 350 |
| TCAAAACACT CTTCTTAATT CAAATGGTAC AATTAATAAA ACGGCTTTTG | 400 |
| AAAAGGCTAA AGCTAAGTTT TTAAATAAAG ATAGACTTGA ATATGGATAT | 450 |
| ATTAGTACTT CATTAATGAA TGTTTCTCAA TTTGCAGGAA GACCAATTAT | 500 |
| TACAAAATTT AAAGTAGCAA AAGGCTCAAA GGCAGGATAT ATTGACCCTA | 550 |
| TTAGTGCTTT TGCAGGACAA CTTGAAATGT TGCTTCCTAG ACATAGTACT | 600 |
| TATCATATAG ACGATATGAG ATTGTCTTCT GATGGTAAAC AAATAATAAT | 650 |
| TACAGCAACA ATGATGGGCA CAGCTATCAA TCCTAAAGAA TTCGTGATGA | 700 |
| ATCCCGCAAA CGCGCAAGGC AGACATACAC CCGGTTAGAG ACTCTAGAGC | 750 |
| TAGAGAAGGA GTTTCACTTC AATCGCTACT TGA | 783 |

pGEX-4TBA-05 Protein Coding Sequence (SEQ ID NO.:4)

```
Gly Ser Ser Arg Val Asp Leu Gln Ala Cys Asn Ala Tyr Ser Ile Asn
  1 5 10 15
Gln Lys Ala Tyr Ser Asn Thr Tyr Gln Glu Phe Thr Asn Ile Asp Gln
                        20 25 30
Ala Lys Ala Trp Gly Asn Ala Gln Tyr Lys Lys Tyr Gly Leu Ser Lys
                35 40 45
Ser Glu Lys Glu Ala Ile Val Ser Tyr Thr Lys Ser Ala Ser Glu Ile
        50 55 60
Asn Gly Lys Leu Arg Gln Asn Lys Gly Val Ile Asn Gly Phe Pro Ser
65 70 75 80
Asn Leu Ile Lys Gln Val Glu Leu Leu Asp Lys Ser Phe Asn Lys Met
                                85 90 95
Lys Thr Pro Glu Asn Ile Met Leu Phe Arg Gly Asp Asp Pro Ala Tyr
                        100 105 110
Leu Gly Thr Glu Phe Gln Asn Thr Leu Leu Asn Ser Asn Gly Thr Ile
                115 120 125
Asn Lys Thr Ala Phe Glu Lys Ala Lys Ala Lys Phe Leu Asn Lys Asp
        130 135 140
Arg Leu Glu Tyr Gly Tyr Ile Ser Thr Ser Leu Met Asn Val Ser Gln
145 150 155 160
Phe Ala Gly Arg Pro Ile Ile Thr Lys Phe Lys Val Ala Lys Gly Ser
                                165 170 175
Lys Ala Gly Tyr Ile Asp Pro Ile Ser Ala Phe Ala Gly Gln Leu Glu
                        180 185 190
Met Leu Leu Pro Arg His Ser Thr Tyr His Ile Asp Asp Met Arg Leu
                195 200 205
Ser Ser Asp Gly Lys Gln Ile Ile Ile Thr Ala Thr Met Met Gly Thr
        210 215 220
Ala Ile Asn Pro Lys Glu Phe Val Met Asn Pro Ala Asn Ala Gln Gly
225 230 235 240
Arg His Thr Pro Gly Thr Arg Leu
                                245
```

Primer 1 Useful to Produce BA-07:

(SEQ ID NO.: 5)

ggatctggtt ccgcgtcata tgtctagagt cgacctg

Primer 2 Useful to Produce BA-07:

(SEQ ID NO.: 6)

Cgcggatcca ttagttctcc ttcttccact tc

pET9a-BA-07 DNA Coding Sequence

pET9a-BA-07 Protein Sequence (SEQ ID NO.:8)

```
Met Ser Arg Val Asp Leu Gln Ala Cys Asn Ala Tyr Ser Ile Asn Gln
  1 5 10 15
Lys Ala Tyr Ser Asn Thr Tyr Gln Glu Phe Thr Asn Ile Asp Gln Ala
                        20 25 30
Lys Ala Trp Gly Asn Ala Gln Tyr Lys Lys Tyr Gly Leu Ser Lys Ser
                35 40 45
Glu Lys Glu Ala Ile Val Ser Tyr Thr Lys Ser Ala Ser Glu Ile Asn
        50 55 60
Gly Lys Leu Arg Gln Asn Lys Gly Val Ile Asn Gly Phe Pro Ser Asn
65 70 75 80
Leu Ile Lys Gln Val Glu Leu Leu Asp Lys Ser Phe Asn Lys Met Lys
                                85 90 95
Thr Pro Glu Asn Ile Met Leu Phe Arg Gly Asp Asp Pro Ala Tyr Leu
                        100 105 110
Gly Thr Glu Phe Gln Asn Thr Leu Leu Asn Ser Asn Gly Thr Ile Asn
                115 120 125
Lys Thr Ala Phe Glu Lys Ala Lys Ala Lys Phe Leu Asn Lys Asp Arg
        130 135 140
Leu Glu Tyr Gly Tyr Ile Ser Thr Ser Leu Met Asn Val Ser Gln Phe
145 150 155 160
Ala Gly Arg Pro Ile Ile Thr Lys Phe Lys Val Ala Lys Gly Ser Lys
                                165 170 175
Ala Gly Tyr Ile Asp Pro Ile Ser Ala Phe Ala Gly Gln Leu Glu Met
                        180 185 190
Leu Leu Pro Arg His Ser Thr Tyr His Ile Asp Asp Met Arg Leu Ser
                195 200 205
Ser Asp Gly Lys Gln Ile Ile Ile Thr Ala Thr Met Met Gly Thr Ala
        210 215 220
Ile Asn Pro Lys Glu Phe Val Met Asn Pro Ala Asn Ala Gln Gly Arg
225 230 235 240
His Thr Pro Gly Thr Arg Leu
                                245
```

An amino acid residue comprises the group —NH—CR₁R₂—CO— when the amino acid residue is located internally in a peptide. The residue is formed from the conesponding amino acid NH₂—CR₁R₂—COOH, wherein R₁ and R₂ are substituents subtended at the central carbon of the amino acid to comprise the remainder of the amino acid, by loss of H₂O to form an amide or a peptide bond with other amino acids, one at the nitrogen and one at the carboxylic acid carbonyl. An amino acid residue at the N-terminus of a peptide comprises the group NH₂—CR₁R₂—CO— in which the carbonyl is bonded by a peptide bond with another amino acid residue in the peptide. An amino acid residue at the C-terminus of a peptide comprises the group —NH—CR₁R₂—COOH in which the nitrogen is bonded by a peptide bond with another amino acid residue in the peptide.

Amino acid residues that can be present in peptide and protein sequences of this invention are sometimes referred to as three letter codes or single letter codes commonly used in the art, which codes comprise: glycine as Gly or G; alanine as Ala or A; valine as Val or V; leucine as Leu or L; isoleucine as Ile or I; methionine as Met or M; phenylalanine as Phe or F; tryptophan Trp or W; proline as Pro or P; serine as Ser or S; threonine as Thr or T; cysteine as Cys or C; tyrosine as Tyr or Y; asparagine as Asn or N; glutamine as Gln or Q; aspartic acid Asp or D; glutamic acid Glu or E; lysine as Lys or K; arginine as Arg or R; and histidine as His or H. Other amino acids that are not essential amino acids can be introduced using methods of peptide synthesis known in the art or by chemical modification such as by acylation (such as by reaction of a lysine epsilon amine group with an active ester comprising a carbonyl group to achieve a bond between the epsilon amine and the carbonyl group), alkylation, urea formation, urethane formation, and the like to add to the peptide chain chemical functional groups containing hydrophobic groups (e.g., C-1 to C-18 alkyl and/or aralkyl, which may be saturated, unsaturated, or contain carbocylic groups such as a proline amide), to add positively charged groups such as quaternary ammonium alkyl groups or basic amino groups that can be protonated at a pH found in a patient with cancer, or both.

In peptides and proteins of this invention, relatively non-polar and hydrophobic amino acid residues can comprise G, A, V, L, I, M, F, W, and P; relatively polar and hydrophilic amino acid residues can comprise S, T, C, Y, N, and Q; anionic and hydrophilic amino acid residues can comprise D and E, wherein in each of D and E a carboxylic acid functional group can be in deprotonated form as an anionic carboxylate; cationic and hydrophilic amino acid residues can comprise K in which the basic epsilon primary amino group can be in protonated form as a cationic ammonium group, H in which the imidazole nitrogen can be in protonated form to provide an imidazolium cationic group, and R which can comprise a protonated amidate group.

Anti-Metastatic Properties of a Pharmaceutical Composition Comprising a Fusion Protein of this Invention

In one aspect, a pharmaceutical composition comprising a fusion protein of this invention can be administered, for example by injection or by a topical application such as by a coating method or other method as described herein to a tissue proximal to or comprising a first tumor in a mammal in need of treatment and can inhibit migration of a metastatic tumor cell in the mammal, the tumor cell originating from a site of the first tumor in the mammal, to a site in healthy or normal tissue of the mammal which is functionally related and proximal to the tissue in which the first tumor resides. For example, a pharmaceutical composition comprising a fusion protein of this invention can be administered to a kidney tissue proximal to or comprising a kidney tumor a mammal and can inhibit migration of a metastatic kidney tumor cell from the tumor in the kidney to healthy tissue in the same kidney in which the first tumor resides.

In another aspect, a pharmaceutical composition comprising a fusion protein of this invention can be administered, for example by injection or by coating or other method as described herein to a tissue proximal to or comprising a first tumor in a mammal in need of treatment, and can inhibit migration of a metastatic tumor cell in the mammal, the tumor cell originating from a site at the first tumor in the mammal, to a site in a healthy or normal tissue or organ in the mammal that is functionally separate from or remote from the tissue in which the first tumor resides. For example, a pharmaceutical composition comprising a fusion protein of this invention be administered to a tissue in the brain comprising a brain tumor, and can inhibit migration of a metastatic brain tumor cell into healthy tissues elsewhere in the body such as liver, spleen or lung tissue.

In another aspect, after administration of a pharmaceutical composition comprising a fusion protein of this invention to a patient in need of treatment, metastatic migration of a malignant tumor cell is prevented or inhibited, and can substantially reduce or completely prevent formation of a secondary tumor and can prevent the spread of malignant cancer in a patient.

Demonstration that a Fusion Protein of this Invention Such as BA-07 can Reduce Cell Motility

The therapeutic effectiveness of a pharmaceutical composition comprising a fusion protein of this invention (such as BA-05) as an anti-metastatic agent can be demonstrated, for example quantitatively, by means of an in vitro two-dimensional cell invasion assay. In one such assay, inhibition of metastatic migration ability of a malignant cell can be measured through the use of purchased Boyden chambers. Boyden chambers have 2 compartments, wherein the upper and lower compartments are separated by a membrane. The extent of cell migration is measured by plating a total number of cells in the upper compartment, and counting the fraction of that total number of cells that migrate to the lower compartment. Growth factors can be added to the lower compartment to enhance cell migration. This model is useful as a model of cancer cell migration in vivo in a mammal. To test the ability of a pharmaceutical composition comprising a fusion protein of this invention (such as BA-07 in sterile phosphate buffered saline that is isotonic with blood of a mammal) to block migration of tumor cells, the composition comprising BA-07 is added at different concentrations of BA-07 to the cancer cells in the upper compartment. The fraction of the total number of cells that migrate to the lower compartment in the presence of fusion protein composition are counted and compared with controls in which the fusion protein is at zero concentration. The number of cancer cells that migrate in a control experiment model such migration in a cancer patient who is not treated with a composition of this invention. The number of cancer cells that migrate in the presence of an aliquot of a composition of this invention model such migration in a cancer patient who is treated with an aliquot of a composition of this invention. The difference between the latter and the control experimental cell migration numbers can be expressed in percent and can range from 100% (i.e., complete inhibition of migration of a metastatic cell) to about 5%, preferably from 100% to about 50%, more preferably from about 100% to about 75%, and most preferably from about 100% to about 90%. A 0% amount can be observed when a first control vehicle is compared with a second control vehicle which may be the same as the first control vehicle. A calculation of this percent is given by solving the expression={(number of cells migrating in the control minus number of cells migrating in the presence of fusion protein) divided by (number of cells migrating in the control)} times 100%.

The therapeutic effectiveness of a pharmaceutical composition comprising a fusion protein of this invention (such as BA-05) as an anti-metastatic agent can be demonstrated at least qualitatively and in one aspect by means of an in vitro three-dimensional cell invasion assay. In one such assay, inhibition of metastatic migration ability of a malignant cell can be measured by the change in the relative ability of a malignant cell to migrate through a MATRIGEL™ matrix after treatment of the cell with a pharmaceutically acceptable formulation of this invention comprising a fusion protein of this invention in a carrier vehicle relative to the ability of the malignant cell to migrate through the MATRIGEL® matrix after treatment with the carrier vehicle as a reference control, the carrier vehicle containing no fusion protein. In one aspect, a fusion protein of this invention can inhibit migration of a metastatic tumor cell in a tissue matrix model to produce an inhibitory change as a reduction in rate of migration of the cell or as a reduction in the distance of migration of the cell in a time period.

The relative change in the distance of migration of a malignant cell through a model matrix is equal to the difference in the distance of migration of a cell in the presence of the fusion protein plus vehicle and the distance of migration of the cell in the presence of a control vehicle in the absence of the fusion protein, the difference divided by the distance of migration of the control vehicle. The relative changes can be expressed in percent and can range from 100% (complete inhibition of migration of a metastatic cell) to about 5%, preferably from 100% to about 50%, more preferably from about 100% to about 75%, and most preferably from about 100% to about 90%. A 0% amount can be observed when a first control vehicle is compared with a second control vehicle which may be the same as the first control vehicle.

In one embodiment, comparison of efficacies of two fusion proteins A and B of this invention, which fusion proteins differ from each other in their amino acid sequence, such as for example in their respective membrane penetration enhancing sequence, may provide different observed percentages of inhibition of migration of a given tumor cell type causes by A and by B. The relative differences (either absolute percentage such as 100% by A versus 80% by B, or qualitative differences such as A is better than B) in inhibition may be the same from tumor type to tumor type or may change from tumor type to tumor type.

In one aspect, a fusion protein of this invention can substantially (100%) inhibit metastatic migration of at least one type of tumor cell.

In another aspect, a fusion protein of this invention can substantially (100%) inhibit metastatic migration of at least two types of tumor cell.

A useful assay is based on the observed ability of an invasive tumor cell to migrate through an artificial basement membrane (MATRIGEL™). In this assay, the change in the ability of different cancer cell types, each with a differing ability to migrate through the MATRIGEL™ in the absence of treatment with a composition of this invention, and hence a differing metastatic invasiveness are evaluated by exposure to a concentration or dose range of a fusion protein of this invention from 0.1 μg/ml to 100 μg/ml. A preferred concentration range is about 0.0001 micrograms of fusion protein per cubic centimeter (cc) of tissue to about 100 micrograms per cubic centimeter of tissue.

Matrigel™ Matrix (BD Biosciences) is a solubulized basement membrane preparation extracted from EHS mouse sarcoma, a tumor rich in ECM proteins. Its major components are laminin, collagen IV, heparan sulfate proteoglycans, and entactin. At room temperature, BD Matrigel™ Matrix polymerizes to produce biologically active matrix material which can mimic mammalian cellular basement membrane, wherein cells can behave in vitro in a manner similar to in vivo conditions. Matrigel™ Matrix can provide a physiologically relevant environment for studies of cell morphology, biochemical function, migration or invasion, and gene expression.

Inhibition of Angiogenesis by a Pharmaceutical Composition Comprising a Fusion Protein of this Invention Such as BA-05, and its Effect on Capillary-Like or Tubule Structures.

In one aspect, a pharmaceutical composition comprising a fusion protein of this invention can be administered, for example by injection or by coating or other method as described herein to a tissue proximal to or comprising a first tumor in a mammal in need of treatment and can inhibit the process of angiogenesis of a metastatic tumor cell or group of tumor cells in the mammal, the tumor cell or group of cells originating from a site of the first tumor in the mammal, to a site in healthy or normal tissue of the mammal which is functionally related and proximal to the tissue in which the first tumor resides. For example, a pharmaceutical composition comprising a fusion protein of this invention can be administered to a kidney tissue proximal to or comprising a kidney tumor a mammal and can inhibit the process of angiogenesis of a metastatic kidney tumor cell from the tumor in the kidney in healthy tissue in the same kidney in which the first tumor resides.

In another aspect, a pharmaceutical composition comprising a fusion protein of this invention can be administered, for example by injection or by coating or other method as described herein to a tissue proximal to or comprising a first tumor in a mammal in need of treatment, and can inhibit the process of angiogenesis associated with growth of a metastatic tumor cell in the mammal, the tumor cell originating from a site at the first tumor in the mammal, to a site in a healthy or normal tissue or organ in the mammal that is functionally separate from or remote from the tissue in which the first tumor resides. For example, a pharmaceutical composition comprising a fusion protein of this invention be administered to a tissue in the brain comprising a brain tumor, and can inhibit angiogenesis of a metastatic brain tumor cell in healthy tissues elsewhere in the body such as in liver, spleen or lung tissue.

In another aspect, after administration of a pharmaceutical composition comprising a fusion protein of this invention to a patient in need of treatment, angiogenesis associated with metastatic formation and growth of a malignant tumor cell can be prevented or inhibited. Administration of a pharmaceutical composition comprising a fusion protein of this invention to a patient in need of treatment, can substantially reduce or completely prevent angiogenesis associated with the formation of a secondary tumor and can prevent the spread and rooting of malignant cancer in a patient.

Formation of new blood vessels by angiogenesis is important in growth of a first tumor and subsequent growth of a second tumor formed from a cell or group of cells of the first tumor by metastasis. Inhibition of angiogenesis by a pharmaceutical composition comprising a fusion protein of this invention such as BA-07 can be evaluated in an in vitro system useful for the study of angiogenesis in the growth of a tumor, i.e., a system comprising cultivation of endothelial cells in the presence of an extract of basement membrane (Matrigel). In the experimental observation conditions, capillary-like structures or tubules associated with angiogenesis or blood vessel capillary formation can be viewed under a microscope. The inhibitory effect of a fusion protein of this invention such as BA-05 on the progress of angiogenesis or on the formation of a tubular capillary network or on the disruption of the process or progress of tumor-associated angiogenesis can be observed by following the disappearance of tubular structures in a Matrigel assay.

In a Matrigel assay, Matrigel (about 12.5 mg/mL) is thawed at about 4° C. The matrix (about 50 uL) is added to each well of a 96 well plate and allowed to solidify for about 10 min at about 37° C. The wells containing solid Matrigel are incubated for about 30 min with HUVEC cells at a concentration of about 15,000 cells per well. When the cells are adhered, medium is removed and replaced by fresh medium supplemented with a fusion protein of this invention such as BA-05 and incubated at 37° C. for about 6 to about 8 hours. Control wells are incubated with medium alone. To analyze the growth, tube formation can be visualized by microscopy at, for example, about 50× magnification. The relative mean length, Yx, of an angiogenesis-derived capillary network observed in an evaluation of a pharmaceutical composition comprising a fusion protein, x, of this invention can be quantified using Northern Eclipse software according to the instructions.

Data from a typical Matrigel assay experiment, for example relating to the effect of a pharmaceutical composition comprising a fusion protein designated as BA-05 on length of an angiogenesis-derived capillary network are summarized in Table 1. These data show that the network formation was inhibited by approximately 13% to about 20% under the dose and formulation conditions used versus the inhibition produced by a control vehicle wherein zero inhibition provides 100% growth. This effect on angiogenesis can be enhanced by using higher doses of fusion protein and by preincubation of the HUVEC cells with BA-05 prior to addition of the cells to Matrigel.

TABLE 1
Anti-angiogenesis effect of a pharmaceutical composition comprising a
fusion protein, BA-05, on the mean length of a capillary network in a
Matrigel matrix assay

| | | Relative mean length of a |
|---|---|---|
| | | capillary network produced |
| | | in the presence of a |
| Mean length | | pharmaceutical composition |
| of a capillary | Relative mean length of a | comprising a fusion protein, |
| network | capillary network produced | BA-05, at a concentration |
| associated with | in the presence of a vehicle | of 10 micrograms per |
| angiogenesis | control | milliter |
| Y1 | 100 | 86.4 |
| Y2 | 100 | 78.2 |
| Y3 | 100 | 86.7 |

Tumor cell antiproliferation activity of pharmaceutical composition comprising a fusion protein of this invention, such as BA-07

Demonstration that a fusion protein of this invention, such as BA-07, can affect multiple aspects of the phenotypes of malignant cells can be shown by monitoring tritiated thymidine incorporation in proliferating and growing cells, wherein tritiated thymidine added to cell culture medium is taken into the cells and becomes part of the thymidine triphosphate pool therein which is used by each cell to synthesize DNA. Tritiated thymidine becomes covalently incorporated into DNA macromolecules in each of the cells. In cells that are not growing or in cells that are undergoing death by apoptosis or by necrosis, tritiated thymidine is either not taken up into the cell or is released into the cell medium upon lysis of the cell. Tritiated thymidine incorporation can be used as an overall measurement of the effect of a fusion protein of this invention such as BA-07 on cell growth, cell division, cell stasis, and cell death. Cell lines in which BA-07 induces a decrease in 3H-thymidin comprise: human endometrial cancer cell line HEC 1B, human colorectal cancer cell line CaCo2, human melanoma cancer cell line SK-MEL-2, and human CNS cancer cell line A-172.

Data in Table 2 illustrate the effects of changes in dosage amounts of a composition comprising a fusion protein of this invention, BA-07, administered to each of eight representative human cancer cell lines on tritiated thymidine incorporation into the eight human cancer cell lines: HEC 1B, Caco-2, SK-MEL-1, HT1080, MCF7, SW480, 293S, and A172. The dose of fusion protein BA-07 administered ranged 50-fold from about 1 micrograms per milliliter to about 10 micrograms per milliliter to about 50 micrograms per milliliter (ug/mL).

TABLE 2
Response data of human tumor cell lines with respect to administration
of a fusion protein, BA-07, as measured by incorporation of tritiated
thymidine

| | Dose of BA-07 in micrograms per milliliter | | |
|---|---|---|---|
| | 50 | 10 | 1 |
| Human Cancer | % growth in the presence of a fusion protein relative to | | |
| Cell Line | that in the presence of a vehicle alone as a control | | |
| HEC 1B | 10 | 13 | 30 |
| Caco-2 | 21 | 17 | 30 |
| SK-MEL-1 | 34 | 30 | 33 |

It was unexpectedly observed that these human tumor cell lines exhibit reduced cell proliferation in the presence of the fusion protein. Table 2 shows the percent of growth compared to a control value of 100%.

Tumor cell lines can be divided into three separate groups with respect to tritiated thymidine incorporation A composition of this invention comprising fusion protein BA-07 exhibits a pronounced effect on cell proliferation in the HEC 1B cell line, which is an endometrial carcinoma cell line, with an inhibition of proliferation related to a 50% inhibitory concentration (IC₅₀) of less than 1 ug/mL. In addition to the inhibition, there is a dose-response effect of increasing inhibition at the higher concentration of BA-07.

In Caco 2 and SK-MEL-1 cell lines, shown in Table 2, a fusion protein exhibits a strong inhibitory effect on cell proliferation as evidenced by lower level of tritiated thymidine incorporation into the cells of each cell line.

Abbreviations used in this disclosure.

ADP adenine dinucleotide phosphate

ATCC American Type culture collection

ADPC3 C3 exotransferase; C3 exoenzyme; C3 transferase

FBS Fetal bovine serum

HEPES HEPES buffer

MMP Matrix metalloproteinase

NAD nicotinamide adenine dinucleotide

NCI National Cancer Institute

PBS phosphate buffered saline

SRB sulforhodamine B

TCA trichloroacetic acid

The invention is further illustrated in various embodiments and aspects by the following non-limiting examples.

### EXAMPLE 1

### General Method Useful to Prepare a Fusion Protein According to this Invention

To demonstrate a method useful to prepare a fusion protein of this invention, an example of an antennapedia sequence added to the C-terminus of the C3 polypeptide is used. The DNA sequence to be added to the C-terminus can be any DNA sequence that will result in addition of at least one amino acid to the C-terminus of C3 polypeptide. The stop codon at the 3′ end of the DNA can be replaced with an EcoR1 site by polymerase chain reaction (PCR) using the primers 5′GAA TTC TTT AGG ATT GAT AGC TGT GCC 3′ (SEQ ID NO: 1) and 5′GGT GGC GAC CAT CCT CCA AAA 3′ (SEQ ID NO: 2). The PCR product can be sub-cloned into a pSTBlue-1 vector (Novagen, Madison, Wis.), then cloned into a pGEX-4T (Amersham Biosciences, Baie d'Urfe, Quebec) vector using BamH I and Not I restriction site. This vector can be called pGEX-4T/C3 and provides a general method to prepare a fusion protein of this invention. An antennapedia sequence useful to add to the 3′ end of C3 in pGEX-4T/C3 can be created by PCR from the pET-3a vector containing the antennapedia sequence (Bloch-Gallego (1993) 120: 485-492; and Derossi (1994) 269: 10444-10450), subcloned into a pSTBlue-1 blunt vector, then cloned into the pGEX-4T/C3, using the restriction sites EcoR I and Sal I, creating pGEX-4T/BA-14. Manipulations of the target plasmid sequence, such as employing nucleases present in plasmid DNA or purchased enzymes that result in new DNA sequences, exonuclease III digestion or site-directed mutagenesis using two synthetic oligonucleotides containing the desired DNA sequence incorporated into the pGEX4T/BA-14, can be used to produce novel DNA sequences that when expressed in an appropriate system produce proteins that can be purified by standard methods such as affinity chromatography or standard chromatography using methods such as ion exchange to separate by charge, size exclusion chromatography to separate by size, and other methods of protein purification. The proteins are tested in assays for ability to permeate cells, and the proteins are tested in assays for their ability to antagonize Rho activity. DNA sequence analysis can be performed on the plasmid sequences that produce responses better than that of C3 exotransferase, each compared as a control. pGEX-4T/BA-14 clone (Seq ID NO: 3) is a currently preferred sequence and provides a protein that is a preferred composition of this invention. An example of a C3-like fusion protein is denoted pGEX-4T/BA-05 (Seq ID NO: 4).

The proteins of the present invention may be prepared from bacterial cell extracts, or through the use of recombinant techniques by transformation, transfection, or infection of a host cell with all or part of a fusion protein-encoding DNA fragment such as a BA-05-encoding DNA fragment) with an antennapedia-derived transport sequence in a suitable expression vehicle.

### EXAMPLE 2

### Preparation of a Fusion Protein, BA-05

The method of example 1 can be used to prepare a fusion protein designated BA-05, which fusion protein contains an amino acid sequence. BA-05 is the name given to the protein made by ligating a cDNA encoding C3 to a cDNA encoding a fusogenic 19-mer peptide.

An example of a C3-like fusion protein is denoted pGEX-4T/BA-05 (Seq ID NO: 4).

This C3-like fusion protein is prepared by the method described to manipulate the antennapedia DNA into the pGEX4T/C3 DNA. Twenty or more C3-like fusion proteins are expressed and are purified as described by the manufacturer (Amersham BioSciences, Baie D′Urfé, Québec). The twenty proteins are examined for ability to inactivate Rho in an in vitro system. Proteins inactivating Rho to a greater extent, as measured by increased neurite outgrowth compared to vehicle control or control glutathione-S-transferase (GST) protein are subjected to further analysis. The products of this process can include proteins such as BA-14, a protein described in the general example, or new fusion proteins produced by the cloning method, which fusion proteins can have properties such as molecular weight and activity in Rho inactivation bioassays different than the fusion protein BA-14 molecule or different from a control of non-fusion protein C3 protein. New fusion proteins can contain a C3 amino acid sequence, but will be altered at the carboxyl terminus due to the method employed.

### EXAMPLE 3

### Preparation of a Fusion Protein, BA-07

The method of example 1 can be used to prepare BA-07, which contains the following amino acid sequence (SEQ ID NO.:8)

```
Met Ser Arg Val Asp Leu Gln Ala Cys Asn Ala Tyr Ser Ile Asn Gln
  1 5 10 15
Lys Ala Tyr Ser Asn Thr Tyr Gln Glu Phe Thr Asn Ile Asp Gln Ala
                        20 25 30
Lys Ala Trp Gly Asn Ala Gln Tyr Lys Lys Tyr Gly Leu Ser Lys Ser
                35 40 45
Glu Lys Glu Ala Ile Val Ser Tyr Thr Lys Ser Ala Ser Glu Ile Asn
        50 55 60
Gly Lys Leu Arg Gln Asn Lys Gly Val Ile Asn Gly Phe Pro Ser Asn
65 70 75 80
Leu Ile Lys Gln Val Glu Leu Leu Asp Lys Ser Phe Asn Lys Met Lys
                                85 90 95
Thr Pro Glu Asn Ile Met Leu Phe Arg Gly Asp Asp Pro Ala Tyr Leu
                        100 105 110
Gly Thr Glu Phe Gln Asn Thr Leu Leu Asn Ser Asn Gly Thr Ile Asn
                115 120 125
Lys Thr Ala Phe Glu Lys Ala Lys Ala Lys Phe Leu Asn Lys Asp Arp
        130 135 140
Leu Glu Tyr Gly Tyr Ile Ser Thr Ser Leu Met Asn Val Ser Gln Phe
145 150 155 160
Ala Gly Arg Pro Ile Ile Thr Lys Phe Lys Val Ala Lys Gly Ser Lys
                                165 170 175
Ala Gly Tyr Ile Asp Pro Ile Ser Ala Phe Ala Gly Gln Leu Glu Met
                        180 185 190
Leu Leu Pro Arg His Ser Thr Tyr His Ile Asp Asp Met Arg Leu Ser
                195 200 205
Ser Asp Gly Lys Gln Ile Ile Ile Thr Ala Thr Met Met Gly Thr Ala
        210 215 220
Ile Asn Pro Lys Glu Phe Val Met Asn Pro Ala Asn Ala Gln Gly Arg
225 230 235 240
His Thr Pro Gly Thr Arg Leu
                                245
```

Two PCR primers are designed to transfer one series of recombinant constructs (BA-05) into the pET-9a vector (Novagen, Madison, Wis.) to create BA-07 protein when expressed in an appropriate expression system: Upper primer: 5′ ggatctggttccgcgtcatatgtctagagtcgacctg 3′ (Seq ID NO: 5) Lower primer: 5′ cgcggatccattagttctccttcttccacttc 3′ (SEQ ID NO: 6). A BamHI site at the 5′ end of Seq ID NO: 6 is ggatccatta; the TGA is replaced by TAAT (atta, in SEQ ID NO: 6).

A program useful to amplify the product using Pfu polymerase comprises: 95° C. 5′1 cycle, then 94° C. 2′® 56° C. 2′® 70° C. 2′ 10 cycles, then 94° C. 2′® 70° C. 3′ 30 cycles and hold at 4° C. A QIAEXII kit (Qiagen) can be used to purify an agarose gel slice containing a desired DNA band. The insert and vector are digested with BamHI and NdeI following the instructions of the manufacturer (New England BioLabs, Beverly, Mass.), purified using agarose gel electrophoresis and a QIAEXII kit (Qiagen), and incubated together overnight with T4 DNA ligase following the manufacturer's directions.

E. coli (DH5alpha, or preferably, XL1-Blue) is transformed with the ligation mixture. The clones can be checked by small scale induction and SDS-PAGE and can be assured by immunoblotting of the crude lysates with anti-C3 antibody. Plasmid DNA is purified, and can be assessed for purity. DNA sequencing can be performed (e.g., by LiCor technology in which the entire strand is sequenced for the full length of the clone).

A first construct prepared in this fashion (pET3a-BA-07, SEQ ID NO:7) matched the theoretical DNA sequence of construct pGEX/BA-05 with a slight change in the 5′ terminus due to the cloning strategy.

A second construct, pET9a-BA-07, can be prepared by subcloning the insert from pET3a-BA-07 into the pET9a vector by cleaving the pET3a construct with BamHI and NdeI (New England BioLabs, Beverly, Mass.) according to the manufacturers instructions. pET9a plasmid DNA can be cleaved with the same enzymes. The insert DNA and the vector DNA can be purified by agarose gel electrophoresis. The insert can be ligated into the new vector using T4 DNA ligase (New England BioLabs, Beverly, Mass.). The ligated DNA can be transformed into DH5alpha cells and DNA can be prepared using QIAGEN mini and maxi kits. Clones can be characterized by restriction digestion and DNA sequencing of the insert in both directions (e.g., by BioS&T, Lachine, Quebec). The construct DNA can be transformed into BL21 (DE3) cells, BL21(DE3)/pLysS cells (Novagen, Madison, Wis.) or another suitable expression system.

### EXAMPLE 4

### General Method for Tritiated Thymidine Uptake as Measure of Cell Proliferation and Useful to Demonstrate that Fusion Protein BA-07 Reduces Proliferation of Cancer Cells

### ³H-Thymidine Incorporation Assays

### Medium and Cell Lines

Cell lines are tested for mycoplasma and found to be negative prior to the initiation of the studies. Cell lines are obtained from ATCC. The line HEC-1B is cultured in E-MEM supplemented with 10% FBS and 1% HEPES. The line Caco-2 is cultured in E-MEM supplemented with 20% FBS, 1% HEPES, 1 mM sodium pyruvate and 0.1 mM of non-essential amino acid. The line SK-MEL-1 is cultured in McCoy's supplemented with 10% FBS and 1% HEPES.

Volumes of 100 μl of each 2× working solution of fusion protein, positive and vehicle controls are plated in triplicate in 96-well microtiter plates containing cells (4×10³/100 μl), yielding a final volume of 200 μl. The plates are placed at 37° C. incubator with 100% humidity and 5% CO₂. After about 54 hours of incubation, a volume of 20 μl of tritiated thymidine (3H-thymidine) (ICN, Montreal, Canada), containing 1.0 μCi, is added to each well. The ³H-thymidine is prepared in RPMI-1640 supplemented with 10% FBS. The cultures are incubated in the same conditions as stated above, for a further 18 hours. At the end of the incubation, the cells are harvested with an automated cell harvester (Tomtec), and the incorporated Counts per minute (cpm) of ³H-thymidine is measured with a microplate scintillation counter (TopCount NXT, Packard).

Demonstration that a fusion protein of this invention, such as BA-07, can affect multiple aspects of the phenotypes of malignant cells can be shown by monitoring tritiated thymidine incorporation in proliferating and growing cells, wherein tritiated thymidine added to cell culture medium is taken into the cells and becomes part of the thymidine triphosphate pool therein which is used by each cell to synthesize DNA. Tritiated thymidine becomes covalently incorporated into DNA macromolecules in each of the cells. In cells that are not growing or in cells that are undergoing death by apoptosis or by necrosis, tritiated thymidine is either not taken up into the cell or is released into the cell medium upon lysis of the cell. Tritiated thymidine incorporation can be used as an overall measurement of the effect of a fusion protein of this invention such as BA-07 on cell growth, cell division, cell stasis, and cell death. Cell lines in which BA-07 induces a decrease in 3H-thymidin comprise: human endometrial cancer cell line HEC 1B, human colorectal cancer cell line CaCo2, human melanoma cancer cell line SK-MEL-2, and human CNS cancer cell line A-172.

Data in Table 2 illustrate the effects of changes in dosage amounts of a composition comprising a fusion protein of this invention, BA-07, administered to each of eight representative human cancer cell lines on tritiated thymidine incorporation into the eight human cancer cell lines: HEC 1B, Caco-2, SK-MEL-1, HT1080, MCF7, SW480, 293S, and A172. The dose of fusion protein BA-07 administered ranged 50-fold from about 1 micrograms per milliliter to about 10 micrograms per milliliter to about 50 micrograms per milliliter (ug/mL).

### EXAMPLE 5

### General Method for Determination of Inhibition of Angiogenesis

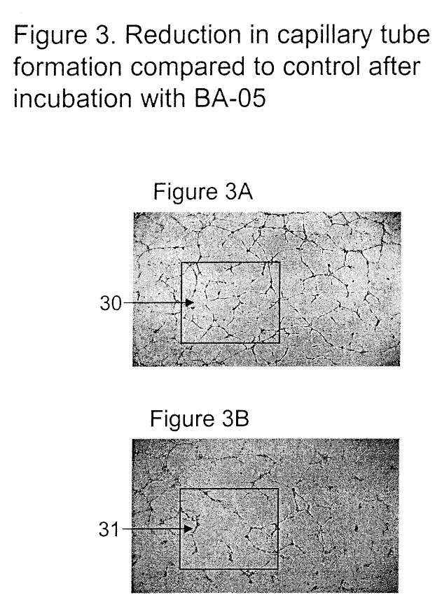

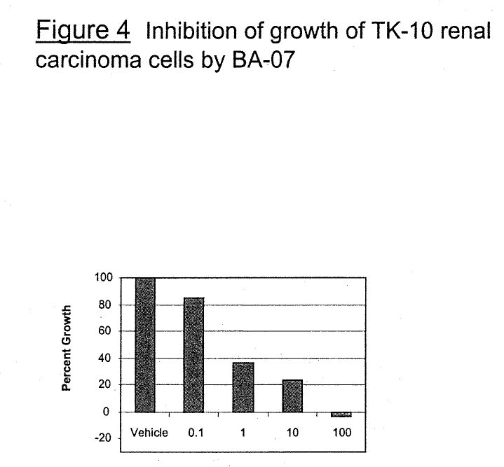

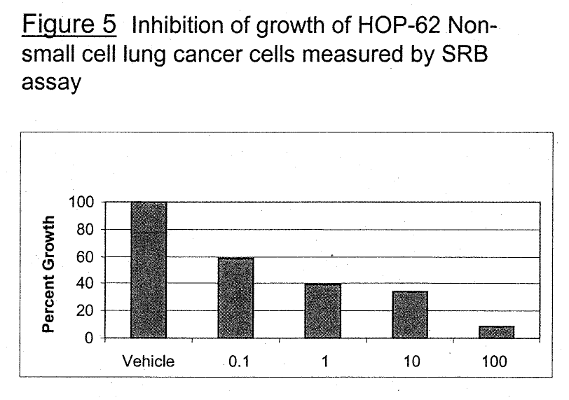

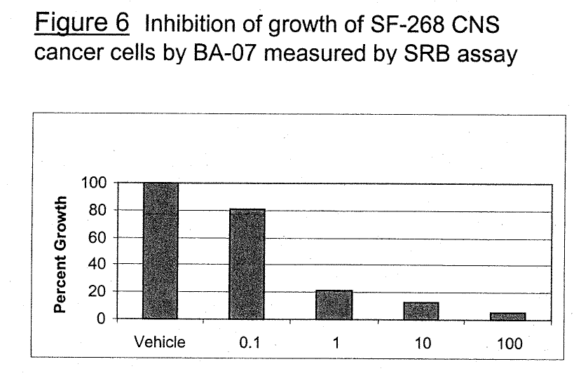

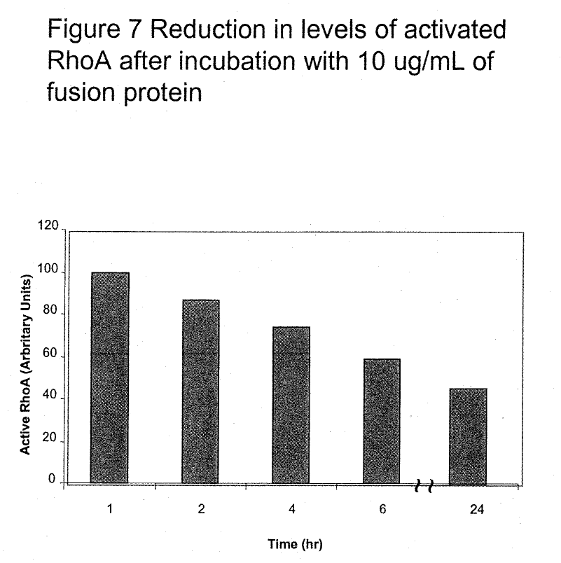

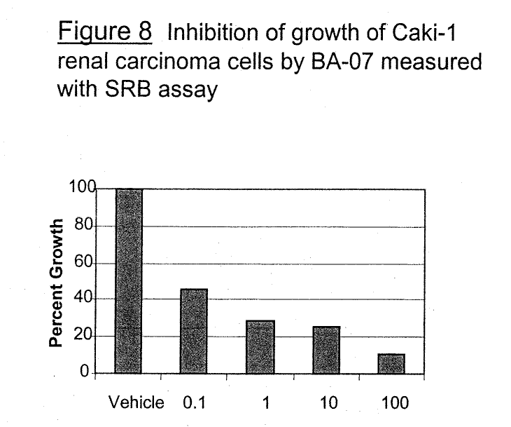

The formation of new blood vessels is studied in a cell culture model by growing endothelial cells in the presence of a matrix of basement membrane (Matigel). Human umbilical vein endothelial cells (HUVEC) are harvested from stock cultures by trypinization, and are resuspended in growth medial consisting of EBM-2 (Clonetics), FBS, hydrocoltisone, hFGF, VEGF, R3-IGF-1, ascorbic acid, hEGF, GA-1000, heparin. Matrigel (12.5 mg/mL) is thawed at 4° C., and 50 mL of Matrigel is added to each well of a 96 well plate, and allowed to solidify for 10 min. at 37° C. Cells in growth medium at a concentration of 15,000 cells/well are added to each well, and are allowed to adhere for 6 hours. The fusion protein, BA-07, is added to the well at about 10 mg/ml, and in other wells PBS is added as control. The cultures are allowed to grow for a further 6 to 8 hours. The growth of tubes can be visualized by microscopy at a magnification of 50×, and the mean length of the capillary network is quantified using Northern Eclipse software. Treatment of the cells in the Matrigel assay with fusion protein BA-07 reduces tube formation (see FIG. 3).

### EXAMPLE 6

### General Method to Demonstrate the Effect of a Fusion Protein on Inhibition of Proliferation of Cancer Cells

### Sulforhodamine B (SRB) Growth Inhibition Assay

A sulforhodamine B (SRB, available from Molecular Probes) protein staining assay for the in vitro measurement of cellular protein content was developed and subsequently adopted for routine use in the NCI in vitro antitumor screening (Skehan et al., 1990). The SRB binds to basic amino acids of cellular protein and calorimetric evaluation provides an estimate of total protein mass which is related to cell number. This assay is based on the assumption that dead cells either lyse and are removed during the procedure, or otherwise do not contribute to the colorimetric end point. The SRB assay might overestimate the surviving fraction of cells.

### Protocol for SRB Assay

These tests are conducted on a NCI 60 cell line panel. Cells are grown in RMPI-L 640 media supplemented with 5% fetal bovine serum and L-glutamine according to ATCC recommendations for each cell line. Cells in logarithmic growth are trypsinized and counted. Cells are inoculated in a 96-well microplate depending on the doubling time of individual cell lines in 100 μL of growth media. The microplates are incubated at 37° C., 5% CO₂ and 100% relative humidity for 24 h to resume exponential growth. After 24 h, two plates of each cell line are fixed in situ with TCA to represent a measurement of the cell population for each cell line at the time of test article addition (T₀). The TCA is removed and the plates are incubated at room temperature for at least 24 h to dry.

A fusion protein of this invention is prepared and stored frozen as a lyophilized powder. It can be reconstituted with sterile water to form a pharmaceutical composition at about 4.42 microgram of fusion protein per microliter in 10 mM sodium phosphate, buffer pH 7.4. For each dose point, serial dilutions of the stock solution are prepared with complete medium containing 50 μg/mL gentamicin to provide fusion protein at 200 μg/mL, 20 μg/mL, 2 μg/mL, 0.2 μg/mL, and 0.02 μg/mL. Aliquots of 100 μL of those test article dilutions are added to the appropriate well already containing 100 μL of medium to achieve the final log dilution series doses for the fusion protein.

After fusion protein (i.e., drug) addition, the microplates are incubated for an additional period at 37° C., 5% CO₂ and 100% relative humidity. The assay is terminated by fixing the protein in the cells to the bottom of the wells using trichloroacetic acid (TCA). The plates are dried, and then 100 μL of SRB solution at 0.4% (w/v) in 1% acetic acid is added to each well. The plates are incubated with the protein-binding stain for 10 min at room temperature.

After staining, unbound dye is removed by washing 1% acetic acid, and the plates are dried. Bound stain is solubilized by adding 200 μL of 10 mM Trizma base while the plates are gently mixed. The amount of dye is measured by reading the optical density with a microplate reader at a wavelength of 515 nm n.

Data is analyzed in an Excel spreadsheet.

T₀=Mean absorbance at the time of fusion protein addition (time 0)
C=Mean absorbance for control (no test article containing drug)
Tᵢ=Mean absorbance for fusion protein article (different dose points in dilution series)

A percentage growth is calculated for each of the test article concentrations:

$$\% Growth = [ \frac{( Ti - To )}{( C - To )} ] \times 100$$

$$for concentrations where {T}_{i} > {T}_{0}$$

$$\% Growth inhibition = [ \frac{( Ti - To )}{( To )} ] \times 100$$

$$for concentrations where {T}_{i} < {T}_{0} .$$

The % growth inhibition can be used to prepare a chart to compare the effect at different doses. The percentage growth plots are plotted, and the points where the dose response curves crossed the PG values of +50, 0, and −50 are used to calculate the GI₅₀, TGI and LC₅₀. GI₅₀, or concentration required to inhibit growth 50% is the relevant parameter for the fusion protein.

### EXAMPLE 7

### Specific Use of SRB Assay to Demonstrate Inhibition of Cell Growth of Human Cancer Cell Lines

TABLE 3
GI50 (concentration for 50% inhibition of cell growth) following
fusion protein treatment measured by SRB assay

| Cell line | Type of Cancer | GI50 (μg/mL) |
|---|---|---|
| Caki-1 | Renal | 0.054 |
| TK-10 | Renal | 0.52 |
| SF-268 | CNS | 0.326 |
| HOP-62 | Non-SCLC | 0.269 |
| NCI-H226 | Non-SCLC | 48.2 |
| HS 578T | Breast | 36.6 |

One fusion protein of this invention, BA_{—}0_, has an effect on 4 of 6 human tumor cell lines tested with ³H-thymidine and an effect on about 10% of the cell lines of the NCI screen. In the SRB test, it appears to have cytostatic properties; growth is inhibited compared to controls but the overall amount of protein does not decrease compared to the amount measured at time zero (Tz). These results agree with in vivo data showing that C3 transferase is not highly toxic to animals. The observed GI₅₀ values are in the nanomolar to micromolar range, given a molecular weight of about 27 kDa for the fusion protein.

### EXAMPLE 8

### Detection of Activated Rho by Pull-Down Assay

NG108 cells are grown in cell culture in the presence of 5% fetal bovine serum (FBS), 1% penicillin-streptomycin (P/S). After the cells settle (3-6 hours at 37° C.), BA-05 is added to the cultures. To lyse the cells, they are washed with ice cold Tris buffered saline (TBS) and are lysed in modified RIPA buffer (50 mM Tris pH 7.2, 1% Triton X-100, 0.5% sodium deoxycholate, 0.1% SDS, 500 mM NaCl, 10 mM MgCl₂, 10 μg/ml leupeptin, 10 μg/ml aprotinin, 1 mM phenylmethyl-sulfonyl fluoride (PMSF)). Cell lysates are clarified by centrifugation at 13,000 g for 10 minutes at 4° C. and kept at minus 80° C. (−80° C.).

Purification of GST-Rho Binding Domain (GST-RBD) is performed with the cell lysates, which are thawed and resuspended in 500 uL of RIPA buffer per 1 million cells. To make the GST-Rho Binding Domain (GST-RBD), bacteria expressing GST-RBD in a PGEX vector are grown in L-broth (LB) with 100 μl/ml amplicillin. Overnight cultures are diluted 1:10 into 3600 ml LB and incubated in a shaking bacterial incubator at 37° C. for 2 hours. Isopropyl-β-D-thiogalactopyranoside (0.5 mM) is then added to the incubating cultures for 2 hours. Bacteria are then collected by centrifugation at 5,000 g for 15 minutes. The pellets are then resuspended in 40 ml lysis buffer (50 mM Tris pH 7.5, 1% Triton-X, 150 mM NaCl, 5 mM MgCl₂, 1 mM DTT, 10 μg/ml leupeptin, 10 μg/ml aprotinin, 1 mM PMSF). After sonication, the lysates are spun at 14,000 rpm for 30 minutes at 4° C.

Frozen cell culture is homogenized in RIPA buffer (50 mM Tris pH 7.2, 1% Triton X-100, 0.5% sodium deoxycholate, 0.1% SDS, 500 mM NaCl, 10 mM MgCl₂, 10 μg/ml leupeptin, 10 μg/ml aprotinin, 1 mM PMSF). The homogenates and cell lysates are clarified by two 10-minute centrifugations at 13,000 g at 4° C. They are then incubated for 50 minutes at 4° C. with GST-RBD coupled to glutathion agarose beads (Sigma, Oakville, Canada). The beads are then washed 4 times and eluted in sample buffer. GTP-bound Rho and total Rho present in tissue homogenates are detected by western blot. The proteins are transferred to nitrocellulose and are probed using a monoclonal RhoA antibody (Santa Cruz, Santa Cruz, Calif.). Bands are visualized with peroxidase-linked secondary antibodies (Promega, Madison, Wyo.) and an HRP based chemiluminescence reaction (Pierce, Rockford, Ill.). Densitometry analysis is performed to quantitate the signal in each band.

### EXAMPLE 9

### Use of Rho Pull-Down Assay as a Diagnostic to Diagnose or Determine which Tumours can Best Respond to Protein Fusion Therapy Using BA-07 as an Example

Biopsy samples of tumours are taken by surgical removal from a tissue in a mammal (e.g., a human patient) to leave residual tissue in the margin of the excised tumor when all of a tumor is removed. The samples are frozen on dry ice or in liquid nitrogen. Samples of excised tissue of approximately 5 mm² are homogenized in 500 uL RIPA buffer (50 mM Tris pH 7.2, 1% Triton X-100, 0.5% sodium deoxycholate, 0.1% SDS, 500 mM NaCl, 10 mM MgCl2, 10 mg/ml leupeptin, 10 mg/ml aprotinin, 1 mM PMSF). The homogenates are clarified by two 10-minute centrifugations at 13,000 g at 4° C. to provide samples for further analysis. The samples are then incubated for 50 minutes at 4° C. with GST-RBD coupled to glutathion agarose beads, prepared as described in example 8. GTP-bound Rho and total Rho present in the tissue homogenates are detected by western blot.

To detect which cells in the biopsy sample have activated Rho, cryostat sections can be prepared. Bacterial lysates of RBD-GST are clarified by centrifugation at 14,000 rpm for 30 minutes at 4° C. Activated Rho is detected by incubating the section with bacterial lysate containing RBD-GST. Rat spinal cord cryosections (about 16 μm thickness) are incubated, after post fixation with 4% PFA, with the bacterial lysate overnight at 4° C. The sections are then washed 3 times is TBS, blocked in 3% BSA for 1 hr at room temperature and incubated with anti-GST antibody (Cell signalling, New England Biolabs, Mississauga, Canada) and with cell-type specific antibodies. In the case of a brain tumour neuron-specific antibody (NeuN) or astrocyte-specific antibody (GFAP) can be used to detect the cell type with activated Rho to aid in tumour diagnosis. Sections are washed in TBS and incubated for 2 hr at room temperature with FITC, Texas Red or Rhodamine conjugated secondary antibodies (Jackson ImmunoResearch, Mississauga, Canada).

### EXAMPLE 10

### General Method to Detect Reduction in Metalloproteinase (MMP) Activity

Metalloproteinase activity is detected by zymography whereby proteolytic activity of enzymes is separated in polyacrylamide gels under non-reducing conditions. To detect metalloproteinase activity the glatinolytic activity in culture media from growth of Caki-1 colon carcinoma cells is detected by gelatin zymography. The Caki-1 cells are incubated with BA-07 at 0.1, 1.0 or 10 μg/ml or buffer as control for 24 hr. An aliquot (25 μL) of the culture media is subjected to SDS/PAGE with 7.5% polyacrylamide containing 1 mg/ml gelatin, and the polypeptides are separated under non-reducing conditions. To assess MMP activity, SDS is removed by incubation for 30 min at room temperature in 2.5% (v/v) Triton X-100. This step is repeated, followed by five rinses with ddH₂0, Next, the gel is incubated for 20 h at 37° C. in a buffer containing 50 mM Tris-HCl, pH 7.6, 0.2 M NaCl, 5 mM CaCl₂, and 0.02% (v/v) Brij-35. The gel is stained with Coomassie Brilliant Blue R-250, and destained. Enzyme activity on the gelatin substrate is detectable as transparent bands in a blue background. The identity of the MMP enzyme with gelatinase activity is assessed with a positive control such as, in these experiments, HT-1080.

### EXAMPLE 11

### Detection of Reduction of Metalloproteinase Activity after Treatment with BA-07

The method of example 10 is employed using the fusion protein BA-07. A reduction in metalloproteinase activity is observed.

### EXAMPLE 12

### Formulation of a Fusion Protein in a Sterile Solution

A therapeutically effective unit dosage amount of a fusion protein of this invention such as BA-07 is dissolved in a unit dosage volume of a sterile isotonic solution such as sterile isotonic PBS to form a unit dosage amount of solution, which is filtered through a 0.2 micron filter under aseptic conditions. The filtrate is collected into a sterile vial under an inert atmosphere (e.g., nitrogen or argon). The vial is then sealed with a sterile septum and crimp cap, and stored at room temperature. The unit dosage amount of solution in the vial containing the fusion protein such as fusion protein BA-07 can be administered to a patient by injection such as by intravenous delivery, infusion, or by injection directly into a tumor site in a mammal such as tumor in a human patient, or by injection into the margins of the site of excision of a tumor in the tissue of a mammal such as a tissue in a human patient.

Two or more vials each containing a unit dosage amount can be prepared in similar fashion, and the unit dosage amounts can be administered by injection over a therapeutically effective time of treatment to a patient in need of treatment. In such fashion, a sequence of unit dosage amount administrations can be made to the tissue of a patient or systemically to a patient. For example, a unit dosage amount of a fusion protein composition can be administered once a day to a patient, or once every two days to a patient, or once a week to a patient. In addition, a therapeutically effective unit dosage amount of a pharmaceutical composition comprising a fusion protein can be administered to a patient having a tumor in a tissue of the patient systemically on one or more occasions before the tumor is excised and/or into the diagnostically identified margins of a tumor in the patient on one or more occasions before the tumor is excised such as by surgical excision, and/or directly into the tumor tissue on one or more occasions before the tumor is excised, and/or systemically on one or more occasions after the tumor is excised, and/or directly into the residual margins of the tumor after the tumor is excised. The number of such repeated unit dosage administrations and the amount of fusion protein per unit dosage form can vary from patient to patient and from tumor type to tumor type and tumor size in order to prevent growth of a second tumor in the presence of a first tumor or after removal of a first tumor.

### EXAMPLE 13

A Lyophilized Formulation

A solution comprising unit dosage amount of a composition of this invention comprising a fusion protein such as BA-07 dissolved in an pharmaceutically acceptable isotonic aqueous medium comprising a pharmaceutically acceptable buffer salt and/or a readily water-soluble pharmaceutically acceptable carbohydrate (preferably a pharmaceutically acceptable non-reducing sugar or a cyclodextrin) is sterile-filtered (e.g. through a 0.2 micron filter) under aseptic conditions, the filtrate is placed in a sterilized vial, the filtrate is frozen, the frozen aqueous solution is lyophilized aseptically at reduced pressure in a pharmaceutically acceptable lyophilizer to leave a dried matrix comprising the fusion protein in the vial, the vial is returned to atmospheric pressure under a sterile inert atmosphere, the vial is sealed with a sterile stopper (e.g. together with a crimp cap). The sealed vial is labeled with its contents and dosage amount and placed in a kit together with a second sealed sterile vial which contains sterilized water for injection in an amount useful to transfer into the first vial containing the lyophilized fusion protein in order to reconstitute the fusion protein matrix to a solution as a unit dosage form. In another embodiment, the fusion protein can be dissolved in a starting volume of aqueous medium which comprises a hypertonic aqueous medium, the solution sterile filtered, the filtrate filled into a vial, and lyophilized to form a dried matrix. This dried matrix can be dissolved or reconstituted in a larger-than-original volume of sterile water, the larger volume sufficient to form an isotonic solution for injection. Alternatively, a hypertonic solution can be used for administration by infusion into a drip bag containing a larger volume of isotonic aqueous medium such that the hypertonic solution is substantially diluted. Optionally, a vial containing a volume of sterile water in an amount suitable to reconstitute the matrix to a unit dosage form is distributed as a kit with the lyophilized protein. Preferably the reconstituted composition comprises an isotonic solution. The fusion protein can be used for intravenous delivery, and/or infusion, and/or direct injection into a tumor with this formulation in a manner similar to that in the previous example.

### EXAMPLE 14

### Formulation in a Polymer

A composition of this invention comprising a fusion protein, such as BA-07, is formulated by blending into a co-polymer of polyglycolic acid (PGA) and polylactic acid (PLA). PGA/PLA co-polymers can degrade 2-6 months after implantation, depending on the ratio of PGA to PLA. In one formulation, PGA/PLA are used and dissolved in a non-denaturing organic solvent at concentrations of 0.5-50%, preferably 1.0-3.0%. The polymer solution can then be spread with a drawdown knife or cast on the surface of a polysaccharide-based film or foam or applied by spray or dip coating or other useful means, and then dried by removal of solvent. Composite mesh such as a mesh comprising a pharmaceutically acceptable dissolvable and/or degradable polymer can be made to incorporate a fusion protein such as BA-07, which will be released as the mesh degrades. The mesh can be implanted in the site of surgical resection of a tumor, and the fusion protein will be released to prevent metastasis and growth of any remaining tumor cells.

### EXAMPLE 15

### General Method to Treat an Excised Tumor Margin

A composition of this invention comprising a fusion protein, such as BA-07, formulated in a pharmaceutically acceptable cream can be used to treat an excision site from the skin. An example is the treatment of malignant melanoma, where such a cream is put on the skin surrounding the excision site of the tumor. In one aspect, such a formulation of a cream containing the fusion protein such as BA-07 can be administered to the skin prior to excision of the tumor and used to treat the tumor between the period of first biopsy and before positive histological diagnosis. The cream when applied to the tumor site can prevent the spread and metastasis of the tumor.

### EXAMPLE 16

### Prevention of a Second Tumour Growing in a Tumour Margin

A composition of this invention comprising a fusion protein, such as BA-07, for example such as an aqueous solution as described above or such as formulated in a surgical adhesive gel, such as a fibrin adhesive or a hydrogel, can be used to treat the area of a surgical resection of a tumor. An example is the treatment of a healthy colon after colonectomy for a colon cancer. The healthy colon tissue that otherwise surrounded the tumor region prior to excision of the tumor can be treated with a fusion protein composition such as BA-07, after removal of the tumor and associated tissue, in a surgical gel such as a fibrin sealant, and will be useful to prevent formation of additional lesions in the residual tissue.

### EXAMPLE 17

### General Method to Demonstrate Preclinical Efficacy in a Mammal

A melanoma cell line is implanted subcutaneously in a first group of nude mice (Charles River Laboratories). Tumors are grown mice of the first group of mice, harvested, and transplanted individually into each mouse (one tumor per mouse) of a second group of mice. A daily injection of a pharmaceutical composition of this invention comprising an effective dose of a fusion protein such as BA-07, which is estimated to be in the range of 10-100 ug/mL of tumor volume, in a pharmaceutically acceptable vehicle is administered to each mouse in the second group of mice. Control animals are injected with vehicle as a control. Tumor growth is measured, and histology performed to measure markers from malignant keratinocytes such as gamma immuno protein 10 (IP10). The composition comprising the fusion protein prevents or substantially inhibits the growth of tumors in the second mice.

### EXAMPLE 18

### Use of a Composition Comprising a Fusion Protein Applied to the Surface of an Implanted Breast Device in the Prevention of Recurrence of Breast Cancer

A therapeutically effective amount of a pharmaceutical composition of this invention comprising a fusion protein is coated onto the surface of a pharmaceutically acceptable breast implant. A tumor is excised from the tissue of a breast in a patient, optionally with co-administration (pre and/or post operative) of a pharmaceutical composition of this invention as described hereinabove. The void created by the excision of the tumor is filled at least in part with the breast implant coated with a pharmaceutical composition comprising a fusion protein, and the wound created by the excision and/or implantation is closed. Growth of a second tumor in the residual tumor margin tissue is substantially inhibited or prevented.

### EXAMPLE 19

### General Method for Preparation of a Fusion Protein

### DNA Sequence of a Representative Fusion Protein, BA-14

Nucleotide Sequence of fusion protein BA-14 (SEQ ID NO: 3)

| | |
|---|---|
| ggatcctcta gagtcgacct gcaggcatgc aatgcttatt ccattaatca aaaggcttat | 60 |
| tcaaatactt accaggagtt tactaatatt gatcaagcaa aagcttgggg taatgctcag | 120 |
| tataaaaagt atggactaag caaatcagaa aaagaagcta tagtatcata tactaaaagc | 180 |
| gctagtgaaa taaatggaaa gctaagacaa aataagggag ttatcaatgg atttccttca | 240 |
| aatttaataa aacaagttga acttttagat aaatctttta ataaaatgaa gacccctgaa | 300 |
| aatattatgt tatttagagg cgacgaccct gcttatttag gaacagaatt tcaaaacact | 360 |
| cttcttaatt caaatggtac aattaataaa acggcttttg aaaaggctaa agctaagttt | 420 |
| ttaaataaag atagacttga attatgatat attagtactt cattaatgaa tgtctctcaa | 480 |
| tttgcaggaa gaccaattat tacacaattt aaagtagcaa aaggctcaaa ggcaggatat | 540 |
| attgacccta ttagtgcttt tcagggacaa cttgaaatgt tgcttcctag acatagtact | 600 |
| tatcatatag acgatatgag attgtcttct gatggtaaac aaataataat tacagcaaca | 660 |
| atgatgggca cagctatcaa tcctaaagaa ttcgtgatgg aatcccgcaa acgcgcaagg | 720 |
| cagacataca cccggtacca gactctagag ctagagaagg agtttcactt caatcgctac | 780 |
| ttgacccgtc ggcgaaggat cgagatcgcc cacgccctgt gcctcacgga gcgccagata | 840 |
| aagatttggt tccagaatcg gcgcatgaag tggaagaagg agaactga | 888 |

Protein Sequence of fusion protein BA-14: (SEQ ID NO.:10)

```
Gly Ser Ser Arg Val Asp Leu Gln Ala Cys Asn Ala Tyr Ser Ile Asn
  1 5 10 15
Gln Lys Ala Tyr Ser Asn Thr Tyr Gln Glu Phe Thr Asn Ile Asp Gln
                        20 25 30
Ala Lys Ala Trp Gly Asn Ala Gln Tyr Lys Lys Tyr Gly Leu Ser Lys
                35 40 45
Ser Glu Lys Glu Ala Ile Val Ser Tyr Thr Lys Ser Ala Ser Glu Ile
        50 55 60
Asn Gly Lys Leu Arg Gln Asn Lys Gly Val Ile Asn Gly Phe Pro Ser
65 70 75 80
Asn Leu Ile Lys Gln Val Glu Leu Leu Asp Lys Ser Phe Asn Lys Met
                                85 90 95
Lys Thr Pro Glu Asn Ile Met Leu Phe Arg Gly Asp Asp Pro Ala Tyr
                        100 105 110
Leu Gly Thr Glu Phe Gln Asn Thr Leu Leu Asn Ser Asn Gly Thr Ile
                115 120 125
Asn Lys Thr Ala Phe Glu Lys Ala Lys Ala Lys Phe Leu Asn Lys Asp
        130 135 140
Arg Leu Glu Tyr Gly Tyr Ile Ser Thr Ser Leu Met Asn Val Ser Gln
145 150 155 160
Phe Ala Gly Arg Pro Ile Ile Thr Gln Phe Lys Val Ala Lys Gly Ser
                                165 170 175
Lys Ala Gly Tyr Ile Asp Pro Ile Ser Ala Phe Gln Gly Gln Leu Glu
                        180 185 190
Met Leu Leu Pro Arg His Ser Thr Tyr His Ile Asp Asp Met Arg Leu
                195 200 205
Ser Ser Asp Gly Lys Gln Ile Ile Ile Thr Ala Thr Met Met Gly Thr
        210 215 220
Ala Ile Asn Pro Lys Glu Phe Val Met Glu Ser Arg Lys Arg Ala Arg
225 230 235 240
Gln Thr Tyr Thr Arg Tyr Gln Thr Leu Glu Leu Glu Lys Glu Phe His
                                245 250 255
Phe Asn Arg Tyr Leu Thr Arg Arg Arg Arg Ile Glu Ile Ala His Ala
                        260 265 270
Leu Cys Leu Thr Glu Arg Gln Ile Lys Ile Trp Phe Gln Asn Arg Arg
                275 280 285
Met Lys Trp Lys Lys Glu Asn
        290 295
```

To demonstrate the method for making a fusion protein of this invention, an example of an antennapedia sequence added to the C-terminus of the C3 polypeptide is useful. A DNA sequence to be added to the C-terminus can be any DNA sequence that will result in addition of at least one amino acid to the C-terminus of a peptide comprising a C3 polypeptide.

First, pGEX2T-C3 plasmid DNA (N. Lamarche, McGill University) is prepared using standard methods. The stop codon at the 3′ end of the DNA can be replaced with an EcoR1 site by polymerase chain reaction (PCR) using the primers 5′GAA TTC TTT AGG ATT GAT AGC TGT GCC 3′ (SEQ ID NO: 1) and 5′GGT GGC GAC CAT CCT CCA AAA 3′ (SEQ ID NO: 2). The PCR product can be sub-cloned into a pSTBlue-1 vector (Novagen, Madison, Wis.), then cloned into a pGEX-4T (Amersham Biosciences, Baie d'Urfe, Quebec) vector using BamH I and Not I restriction site. This vector can be called pGEX-4T/C3 and provides a general method to prepare a fusion protein of this invention. An antennapedia sequence useful to add to the 3′ end of C3 in pGEX-4T/C3 can be created by PCR from the pET-3a vector containing the antennapedia sequence (Bloch-Gallego (1993) 120: 485-492; and Derossi (1994) 269: 10444-10450), subcloned into a pSTBlue-1 blunt vector, then cloned into the pGEX-4T/C3, using the restriction sites EcoR I and Sal I, creating pGEX-4T/BA-14.

Nucleotide Sequence of BA-_ (SEQ ID NO: 3) [remove C3APL]

| | |
|---|---|
| ggatcctcta gagtcgacct gcaggcatgc aatgcttatt ccattaatca aaaggcttat | 60 |
| tcaaatactt accaggagtt tactaatatt gatcaagcaa aagcttgggg taatgctcag | 120 |
| tataaaaagt atggactaag caaatcagaa aaagaagcta tagtatcata tactaaaagc | 180 |
| gctagtgaaa taaatggaaa gctaagacaa aataagggag ttatcaatgg atttccttca | 240 |
| aatttaataa aacaagttga acttttagat aaatctttta ataaaatgaa gacccctgaa | 300 |
| aatattatgt tatttagagg cgacgaccct gcttatttag gaacagaatt tcaaaacact | 360 |
| cttcttaatt caaatggtac aattaataaa acggcttttg aaaaggctaa agctaagttt | 420 |
| ttaaataaag atagacttga atatggatat attagtactt cattaatgaa tgtctctcaa | 480 |
| tttgcaggaa gaccaattat tacacaattt aaagtagcaa aaggctcaaa ggcaggatat | 540 |
| attgacccta ttagtgcttt tcagggacaa cttgaaatgt tgcttcctag acatagtact | 600 |
| tatcatatag acgatatgag attgtcttct gatggtaaac aaataataat tacagcaaca | 660 |
| atgatgggca cagctatcaa tcctaaagaa ttcgtgatgg aatcccgcaa acgcgcaagg | 720 |
| cagacataca cccggtacca gactctagag ctagagaagg agtttcactt caatcgctac | 780 |
| ttgacccgtc ggcgaaggat cgagatcgcc cacgccctgt gcctcacgga gcgccagata | 840 |
| aagatttggt tccagaatcg gcgcatgaag tggaagaagg agaactga | 888 |

Protein Sequence of Fusion Protein BA-14: (SEQ ID NO. 10)

```
Gly Ser Ser Arg Val Asp Leu Gln Ala Cys Asn Ala Tyr Ser Ile Asn
  1 5 10 15
Gln Lys Ala Tyr Ser Asn Thr Tyr Gln Glu Phe Thr Asn Ile Asp Gln
                        20 25 30
Ala Lys Ala Trp Gly Asn Ala Gln Tyr Lys Lys Tyr Gly Leu Ser Lys
                35 40 45
Ser Glu Lys Glu Ala Ile Val Ser Tyr Thr Lys Ser Ala Ser Glu Ile
        50 55 60
Asn Gly Lys Leu Arg Gln Asn Lys Gly Val Ile Asn Gly Phe Pro Ser
65 70 75 80
Asn Leu Ile Lys Gln Val Glu Leu Leu Asp Lys Ser Phe Asn Lys Met
                                85 90 95
Lys Thr Pro Glu Asn Ile Met Leu Phe Arg Gly Asp Asp Pro Ala Tyr
                        100 105 110
Leu Gly Thr Glu Phe Gln Asn Thr Leu Leu Asn Ser Asn Gly Thr Ile
                115 120 125
Asn Lys Thr Ala Phe Glu Lys Ala Lys Ala Lys Phe Leu Asn Lys Asp
        130 135 140
Arg Leu Glu Tyr Gly Tyr Ile Ser Thr Ser Leu Met Asn Val Ser Gln
145 150 155 160
Phe Ala Gly Arg Pro Ile Ile Thr Gln Phe Lys Val Ala Lys Gly Ser
                                165 170 175
Lys Ala Gly Tyr Ile Asp Pro Ile Ser Ala Phe Gln Gly Gln Leu Glu
                        180 185 190
Met Leu Leu Pro Arg His Ser Thr Tyr His Ile Asp Asp Met Arg Leu
                195 200 205
Ser Ser Asp Gly Lys Gln Ile Ile Ile Thr Ala Thr Met Met Gly Thr
        210 215 220
Ala Ile Asn Pro Lys Glu Phe Val Met Glu Ser Arg Lys Arg Ala Arg
225 230 235 240
Gln Thr Tyr Thr Arg Tyr Gln Thr Leu Glu Leu Glu Lys Glu Phe His
                                245 250 255
Phe Asn Arg Tyr Leu Thr Arg Arg Arg Arg Ile Glu Ile Ala His Ala
                        260 265 270
Leu Cys Leu Thr Glu Arg Gln Ile Lys Ile Trp Phe Gln Asn Arg Arg
                275 280 285
Met Lys Trp Lys Lys Glu Asn
        290 295
```

The fusion proteins of the present invention may be prepared from bacterial cell extracts, or through the use of recombinant techniques by transformation, transfection, or infection of a host cell with all or part of a fusion protein-encoding DNA fragment such as a BA-05-encoding DNA fragment) with an antennapedia-derived transport sequence in a suitable expression vehicle.

### EXAMPLE 20

### Preparation of a Fusion Protein, BA-05

An example of a C3-like fusion protein is denoted pGEX-4T/BA-05 (Seq ID NO: 4).

BA-05 is the name given herein to a protein made by ligating a cDNA encoding C3 to a cDNA encoding a fusogenic 19-mer peptide. The method of example 19 can be used to prepare a fusion protein, BA-05, which contains the following amino acid sequence:

pGEX-4TBA-05 Protein Coding Sequence

```
Gly Ser Ser Arg Val Asp Leu Gln Ala Cys Asn Ala Tyr Ser Ile Asn
  1 5 10 15
Gln Lys Ala Tyr Ser Asn Thr Tyr Gln Glu Phe Thr Asn Ile Asp Gln
                        20 25 30
Ala Lys Ala Trp Gly Asn Ala Gln Tyr Lys Lys Tyr Gly Leu Ser Lys
                35 40 45
Ser Glu Lys Glu Ala Ile Val Ser Tyr Thr Lys Ser Ala Ser Glu Ile
        50 55 60
Asn Gly Lys Leu Arg Gln Asn Lys Gly Val Ile Asn Gly Phe Pro Ser
65 70 75 80
Asn Leu Ile Lys Gln Val Gln Leu Leu Asp Lys Ser Phe Asn Lys Met
                                85 90 95
Lys Thr Pro Glu Asn Ile Met Leu Phe Arg Gly Asp Asp Pro Ala Tyr
                        100 105 110
Leu Gly Thr Glu Phe Gln Asn Thr Leu Leu Asn Ser Asn Gly Thr Ile
                115 120 125
Asn Lys Thr Ala Phe Glu Lys Ala Lys Ala Lys Phe Leu Asn Lys Asp
        130 135 140
Arg Leu Glu Tyr Gly Tyr Ile Ser Thr Ser Leu Met Asn Val Ser Gln
145 150 155 160
Phe Ala Gly Arg Pro Ile Ile Thr Gln Phe Lys Val Ala Lys Gly Ser
                                165 170 175
Lys Ala Gly Tyr Ile Asp Pro Ile Ser Ala Phe Gln Gly Gln Leu Glu
                        180 185 190
Met Leu Leu Pro Arg His Ser Thr Tyr His Ile Asp Asp Met Arg Leu
                195 200 205
Ser Ser Asp Gly Lys Gln Ile Ile Ile Thr Ala Thr Met Met Gly Thr
        210 215 220
Ala Ile Asn Pro Lys Glu Phe Val Met Glu Ser Arg Lys Arg Ala Arg
225 230 235 240
Gln Thr Tyr Thr Arg Tyr Gln Thr Leu Glu Leu Glu Lys Glu Phe His
                                245 250 255
Phe Asn Arg Tyr Leu Thr Arg Arg Arg Arg Ile Glu Ile Ala His Ala
                        260 265 270
Leu Cys Leu Thr Glu Arg Gln Ile Lys Ile Trp Phe Gln Asn Arg Arg
                275 280 285
Met Lys Trp Lys Lys Glu Asn
        280 285
```

This C3-like fusion protein is prepared by the method described to manipulate an antennapedia DNA into the pGEX4T/C3 DNA, producing pGEX4T/BA-14. A clone with a frameshift mutation is selected, and the protein is made and tested. When cultures test positive despite the presence of a mutation, the plasmid DNA is resequenced to confirm the mutation. The new clone is herein called BA-05. To confirm the sequence of C3APLT, the coding sequence from both strands are sequenced. The sequence for this clone is given in Examples herein (nucleotide sequence of BA-05; SEQ ID NO: 9, amino acid sequence of BA-05; SEQ ID NO:4).

Another method useful to make BA-05 is to prepare pGEX-4T/BA-14, then use the technique of site-directed mutagenesis using two complementatry oligonucleotide primers such as (SEQ ID No. 11) 5′ CCTAAAGAATTCGTGATGAATCCCGCAAACGCGCA 3′ and SEQ ID No. 12 5′ TGCGCGTTTGCGGGATTCATCACGAATTCTTTAGG 3′) containing a 1 basepair deletion in the pGEX4T-BA14 DNA. A QuikChange kit (Statragene, LaJolla, Calif.) is used to incorporate the deletion using extension of the primers in the presence of nucleotides. The following cycle of temperatures is useful for preparation of BA-05: 1 cycle for 30 s at 95 C, then 18 cycles of 95 C for 30 s, 55 C for 1 min, and 68 C for 10.5 min. The DNA is then treated with the restriction enzyme DpnI as described by the manufacturer. A portion of the reaction is then transformed into E. coli DH5alpha or XL1-Blue. Individual colonies of E. coli are isolated on agar plates containing selective antibiotic, and grown in LB medium+ampicillin. DNA is isolated using a MidiPrep Kit (Qiagen). The DNA of 5 clones is sequenced and the sequence change is confirmed. Protein is expressed from the DNA and purified as described in Lehmann et al., 1999. The purified protein can be used as a Rho antagonist in biological systems.

To prepare recombinant BA-05 (SEQ ID NO.: _) the plasmids containing the corresponding cDNA (pGEX-4T/BA-05) are transformed into bacteria, strain XL-1 blue competent E. coli. The bacteria are grown in L-broth (10 g/L Bacto-Tryptone, 5 g/L Yeast Extract, 10 g/L NaCl) with ampicillin at 50 ug/ml (BMC-Roche), in a shaking incubator for 1 hr at 37° C. and 300 rpm. Isopropyl .beta.-D-thiogalactopyranoside (IPTG), (Gibco) is added to a final concentration of 0.5 mM to induce the production of recombinant protein and the culture is grown for a further 6 hours at 37° C. and 250 rpm. Bacteria pellets ware obtained by centrifugation in 250 ml centrifuge bottles at 7000 rpm for 6 minutes at 4.degree. C. Each pellet is re-suspended in 10 ml of Buffer A (50 mM Tris, pH 7.5, 50 mM NaCl, 5 mM MgCl.₂, 1 mM DTT) plus 1 mM PMSF. All resuspended pellets are pooled and transferred to a 100 ml plastic beaker on ice. The remaining Buffer A with PMSF is added to the pooled sample. The bacteria sample is sonicated 6×20 seconds using a Branson Sonifier 450 probe sonicator. Both the bacteria and probe are cooled on ice 1 minute between sonications. The sonicate is centrifuged in a Sorvall SS-34 rotor at 16,000 rpm for 12 minutes at 4° C. to clarify the supernatant. The supernatant is transferred into fresh SS-34 tubes and re-spun at 12,000 rpm for 12 minutes at 4° C. Up to 20 ml of Glutathione-agarose beads (Sigma) are added to the cleared lysate and placed on a rotating plate for 2 to 3 hours. The beads are washed 4 times with buffer B, (Buffer A, NaCl is 150 mM, no PSMF) then 2 times with Buffer C (Buffer B+2.5 mM CaCl₂). The final wash is poured out till the beads create a thick slurry. To remove the glutathione S transferase sequence from the recombinant protein, 20 U of Thrombin (Bovine, Plasminogen-free, Calbiochem) is added, the beads are left on a rotator overnight at 4° C. After cleavage with thrombin the beads are loaded into an empty 20 ml column. Approximately 20 aliquots of 1 ml are collected by elution with PBS. Samples of each aliquot of 0.5 ul are spotted on nitrocellulose and stained with Amido Black to determine the protein peak. Aliquots containing fusion proteins are pooled and 100 microliters of p-aminobenzamidine agarose beads (Sigma) are added and left mixing for 45 minutes at 4° C. This last step removes the thrombin from the recombinant protein sample. The recombinant protein is centrifuged to remove the beads and then concentrated using a centriprep-10 concentrator (Amicon). The concentrated recombinant protein is desalted with a PD-10 column (Pharmacia, containing Sephadex G-25M) and ten 0.5 ml aliquots are collected. A dot-blot is done on these samples to determine the protein peak, and the appropriate aliquots pooled, filter-sterilized, and stored at −80° C. A protein assay (DC assay, Biorad) is used to determine the concentration of recombinant protein. Purity of the sample is determined by SDS-PAGE, and bioactivity bioassay with NG-108 cells.

The products of this process can include fusion proteins such as BA-14 as described in the general example, or new fusion proteins produced by the cloning method that have properties such as molecular weight and activity in Rho inactivation bioassays different than the BA-14 molecule or control C3 protein, such as BA-05. These new fusion proteins will contain the C3 sequence and will be altered at the carboxyl terminus due to the method employed.

### EXAMPLE 21

### Preparation of Fusion Protein BA-07

The method of example 1 can be used to prepare a fusion protein BA-07 which contains the following amino acid sequence (SEQ ID NO. 8):

```
Met Ser Arg Val Asp Leu Gln Ala Cys Asn Ala Tyr Ser Ile Asn Gln
  1 5 10 15
Lys Ala Tyr Ser Asn Thr Tyr Gln Glu Phe Thr Asn Ile Asp Gln Ala
                        20 25 30
Lys Ala Trp Gly Asn Ala Gln Tyr Lys Lys Tyr Gly Leu Ser Lys Ser
                35 40 45
Glu Lys Glu Ala Ile Val Ser Tyr Thr Lys Ser Ala Ser Glu Ile Asn
        50 55 60
Gly Lys Leu Arg Gln Asn Lys Gly Val Ile Asn Gly Phe Pro Ser Asn
65 70 75 80
Leu Ile Lys Gln Val Glu Leu Leu Asp Lys Ser Phe Asn Lys Met Lys
                                85 90 95
Thr Pro Glu Asn Ile Met Leu Phe Arg Gly Asp Asp Pro Ala Tyr Leu
                        100 105 110
Gly Thr Glu Phe Gln Asn Thr Leu Leu Asn Ser Asn Gly Thr Ile Asn
                115 120 125
Lys Thr Ala Phe Glu Lys Ala Lys Ala Lys Phe Leu Asn Lys Asp Arg
        130 135 140
Leu Glu Tyr Gly Tyr Ile Ser Thr Ser Leu Met Asn Val Ser Gln Phe
145 150 155 160
Ala Gly Arg Pro Ile Ile Thr Lys Phe Lys Val Ala Lys Gly Ser Lys
                                165 170 175
Ala Gly Tyr Ile Asp Pro Ile Ser Ala Phe Ala Gly Gln Leu Glu Met
                        180 185 190
Leu Leu Pro Arg His Ser Thr Tyr His Ile Asp Asp Met Arg Leu Ser
                195 200 205
Ser Asp Gly Lys Gln Ile Ile Ile Thr Ala Thr Met Met Gly Thr Ala
        210 215 220
Ile Asn Pro Lys Glu Phe Val Met Asn Pro Ala Asn Ala Gln Gly Arg
225 230 235 240
His Thr Pro Gly Thr Arg Leu
                                245
```

Two PCR primers are designed to transfer one series of recombinant constructs (BA-05) into the pET-9a vector (Novagen, Madison, Wis.) to create BA-07 protein when expressed in an appropriate expression system: Upper primer: 5′ GGATCTGGTTCCGCGTCATATGTCTAGAGTCGACCTG 3′ (Seq ID NO: 5) Lower primer: 5′ CGCGGATCCATTAGTTCTCCTTCTTCCACTTC 3′ (SEQ ID NO: 6). A BamHI site at the 5′ end of Seq ID NO: 6 is ggatccatta; the TGA is replaced by TAAT (atta, in SEQ ID NO: 6).

A program useful to amplify the product using Pfu polymerase comprises: 95° C. 5′ 1 cycle, then 94° C. 2′→56° C. 2′→70° C. 2′ 10 cycles, then 94° C. 2′→70° C. 3′ 30 cycles and hold at 4° C. A QIAEXII kit (Qiagen) can be used to purify an agarose gel slice containing a desired DNA band. The insert and vector are digested with BamHI and NdeI following the instructions of the manufacturer (New England BioLabs, Beverly, Mass.), purified using agarose gel electrophoresis and a QIAEXII kit (Qiagen), and incubated together overnight with T4 DNA ligase following the manufacturer's directions.

E. coli (DH5alpha, or preferably, XL1-Blue) is transformed with the ligation mixture. The clones can be checked by small-scale induction and SDS-PAGE and can be assured by immunoblotting of the crude lysates with anti-C3 antibody. Plasmid DNA is purified, and can be assessed for purity. DNA sequencing can be performed (e.g., by LiCor technology in which the entire strand is sequenced for the full length of the clone).

A first construct is prepared in this fashion (pET3a-BA-07, SEQ ID NO:7) and acceptably matches the theoretical DNA sequence of construct pGEX/BA-05 with a slight change in the 5′ terminus due to the cloning strategy.

A second construct, pET9a-BA-07, can be prepared by subcloning the insert from pET3a-BA-07 into the pET9a vector by cleaving the pET3a construct with BamHI and NdeI (New England BioLabs, Beverly, Mass.) according to the manufacturers instructions. pET9a plasmid DNA can be cleaved with the same enzymes. The insert DNA and the vector DNA can be purified by agarose gel electrophoresis. The insert can be ligated into the new vector using T4 DNA ligase (New England BioLabs, Beverly, Mass.). The ligated DNA can be transformed into DH5alpha cells and DNA can be prepared using QIAGEN mini and maxi kits. Clones can be characterized by restriction digestion and DNA sequencing of the insert in both directions (e.g., BioS&T, Lachine, Quebec). The construct DNA can be transformed into BL21 (DE3) cells, BL21(DE3)/pLysS cells (Novagen, Madison, Wis.) or another suitable expression system.

### EXAMPLE 22

### General Method for Tritiated Thymidine Uptake as Measure of Cell Proliferation

### ³H-Thymidine Incorporation Assays

Cell lines are tested for mycoplasma and found to be negative prior to the initiation of the studies. Cell lines are obtained from American Type Culture Collection (ATCC) (Rockville, Md.). The line HEC-1B is cultured in Eagles Minimal Essential Medium (E-MEM) supplemented with 10% fetal bovine serum (FBS) and 1% HEPES. The line Caco-2 is cultured in E-MEM supplemented with 20% FBS, 1% HEPES, 1 mM sodium pyruvate and 0.1 mM of non-essential amino acid. The line SK-MEL-1 is cultured in Mc Coy's minimal medium supplemented with 10% FBS and 1% HEPES. Volumes of 100 μl of each 2× working solution of C3-07, positive and vehicle controls are plated in triplicate in 96-well microtiter plates containing cells (4×10³/100 μl), yielding a final volume of 200 μl. The plates were placed at 37° C. incubator with 100% humidity and 5% CO₂. After 54 hours of incubation, a volume of 20 μl of tritiated thymidine (H-thymidine) (ICN, Montreal, Canada), containing 1.0 μCi, is added to each well. The ³H-thymidine is prepared in RPMI-1640 medium supplemented with 10% FBS. The cultures are incubated in the same conditions as stated above, for a further 18 hours.

At the end of the incubation, the cells are harvested with an automated cell harvester (Tomtec), and the incorporated counts per minute (cpm) of ³H-thymidine is measured with a microplate scintillation counter (TopCount NXT, Packard). Values from the wells treated with the BA-07 fusion protein are compared to values of the vehicle control. Data is graphed with counts per minute (cpm) on the y axis and the dose of fusion protein on the X axis.

What is claimed is:

1. A method of inhibition of uncontrolled proliferation and spreading or migration of a metastatic neoplastic cell of a cancer in a mammal, comprising administration to the mammal of a therapeutically effective amount of a composition comprising a polypeptide comprising an amino acid sequence of a transport agent covalently linked to an amino acid sequence of an active agent region, said amino acid sequence of said active agent region consisting of ADP-ribosyl transferase C3 or a fragment thereof retaining an ADP-ribosyl transferase activity, said amino acid sequence of said transport agent facilitating uptake of the active agent by a receptor independent mechanism and being selected from the group consisting of a subdomain of HIV Tat protein, a homeodomain of antennapedia, a proline-rich region and a Histidine tag, or a functional analog thereof, wherein said metastatic neoplastic cell is selected from the group consisting of colorectal cancer cell, melanoma cell and glioblastoma cell.

2. A method of inhibition of growth of a tumor from a malignant cell in a host tissue in a mammal comprising administration to the mammal of a therapeutically effect amount of a composition comprising a polypeptide comprising an amino acid sequence of a transport agent covalently linked to an amino acid sequence of an active agent region, said amino acid sequence of said active agent region consisting of ADP-ribosyl transferase C3 or a fragment thereof retaining an ADP-ribosyl transferase activity, said amino acid sequence of said transport agent facilitating uptake of the active agent by a receptor independent mechanism and being selected from the group consisting of a subdomain of HIV Tat protein, a homeodomain of antennapedia, a proline-rich region and a Histidine tag, or a functional analog thereof, wherein said malignant cell is selected from the group consisting of colorectal cancer cell, melanoma cell and glioblastoma cell.

3. The method of claim 1, wherein said amino acid sequence of said active agent region is selected from the group consisting of BA-05 (SEQ ID NO:4), BA-07 (SEQ ID NO:8) and BA-14 (SEQ ID NO:10).

4. The method of claim 1, wherein the therapeutically effective amount is about 0.001 micrograms per cc to about 50 micrograms per cc of tissue.

5. The method of claim 1, wherein the therapeutically effective amount is about 0.0001 micrograms of fusion protein per cubic centimeter (cc) of tissue to about 100 micrograms per cubic centimeter of tissue.

6. The method of claim 1, wherein the therapeutically effective amount is about 1 micrograms per milliliter to about 10 micrograms per milliliter to about 50 micrograms per milliliter.

7. The method of claim 1, wherein the administration is by local injection.

8. The method of claim 1, wherein the administration is selected from the group consisting of intrarticular, intraocular, intranasal, intraneural, intradermal, intraosteal, sublingual, oral, topical, intravesical, intrathecal, intravenous, intraperitoneal, intracranial, intramuscular, subcutaneous, inhalation, atomization and inhalation, application directly into a tumor, application directly into a disease site, application directly on or into the margins remaining after resection of a tumor, enteral, enteral together with a gastroscopic procedure, and ECRP.

9. The method of claim 1, wherein the polypeptidic cell-membrane transport moiety comprises a peptide containing from about 5 to about 50 amino acids.

10. The method of claim 1, wherein the functional analog comprises a protein exhibiting activity in the range of 50% to 500% of that of wild type Clostridium botulinum Ce exotransferase.
